(19) United States
(12) Patent Application Publication
(10) Pub. No.: US 2012/0253200 A1
(43) Pub. Date: Oct. 4, 2012

(54) LOW-COST IMAGE-GUIDED NAVIGATION AND INTERVENTION SYSTEMS USING COOPERATIVE SETS OF LOCAL SENSORS

(75) Inventors: Philipp Jakob STOLKA, Baltimore, MD (US); Emad Moussa BOCTOR, Baltimore, MD (US)

(73) Assignee: The Johns Hopkins University, Baltimore, MD (US)

(21) Appl. No.: 13/476,838

(22) Filed: May 21, 2012

Related U.S. Application Data
(63) Continuation of application No. PCT/US2010/057482, filed on Nov. 19, 2010.
(60) Provisional application No. 61/262,735, filed on Nov. 19, 2009.

(51) Int. Cl. A61B 1/04; A61B 8/00; H04N 7/18

(52) U.S. Cl. 600/459; 348/65; 600/109; 348/E07.085

(57) ABSTRACT

An augmentation device for an imaging system has a bracket structured to be attachable to an imaging component, and a projector attached to the bracket. The projector is arranged and configured to project an image onto a surface in conjunction with imaging by the imaging system. A system for image-guided surgery has an imaging system, and a projector configured to project an image or pattern onto a region of interest during imaging by the imaging system. A capsule imaging device has an imaging system, and a local sensor system. The local sensor system provides information to reconstruct positions of the capsule endoscope free from external monitoring equipment.

### CROSS-REFERENCE OF RELATED APPLICATION

This application claims priority to U.S. Provisional Application No. 61/262,735 filed Nov. 19, 2009, the entire contents of which are hereby incorporated by reference.

### BACKGROUND

1. Field of Invention

The field of the currently claimed embodiments of this invention relate to imaging devices and to augmentation devices for these imaging devices, and more particularly to such devices that have one or more of a camera, one or more of a projector, and/or a set of local sensors for observation and imaging of, projecting onto, and tracking within and around a region of interest.

2. Discussion of Related Art

Image-guided surgery (IGS) can be defined as a surgical or intervention procedure where the doctor uses indirect visualization to operate, i.e. by employing imaging instruments in real time, such as fiber-optic guides, internal video cameras, flexible or rigid endoscopes, ultrasonography etc. Most image-guided surgical procedures are minimally invasive. IGS systems allow the surgeon to have more information available at the surgical site while performing a procedure. In general, these systems display 3D patient information and render the surgical instrument in this display with respect to the anatomy and a preoperative plan. The 3D patient information can be a preoperative scan such as CT or MRI to which the patient is registered during the procedure, or it can be a real-time imaging modality such as ultrasound or fluoroscopy. Such guidance assistance is particularly crucial for minimally invasive surgery (MIS), where a procedure or intervention is performed either through small openings in the body or percutaneously (e.g. in ablation or biopsy procedures). MIS techniques provide for reductions in patient discomfort, healing time, risk of complications, and help improve overall patient outcomes.

Minimally invasive surgery has improved significantly with computer-integrated surgery (CIS) systems and technologies. CIS devices assist surgical interventions by providing pre- and intra-operative information such as surgical plans, anatomy, tool position, and surgical progress to the surgeon, helping to extend his or her capabilities in an ergonomic fashion. A CIS system combines engineering, robotics, tracking and computer technologies for an improved surgical environment [Taylor R H, Lavallee S, Burdea G C, Mosges R, “Computer-Integrated Surgery Technology and Clinical Applications,” MIT Press, 1996]. These technologies offer mechanical and computational strengths that can be strategically invoked to augment surgeons' judgment and technical capability. They enable the “intuitive fusion” of information with action, allowing doctors to extend minimally invasive solutions into more information-intensive surgical settings.

In image-guided interventions, the tracking and localization of imaging devices and medical tools during procedures are exceptionally important and are considered the main enabling technology in IGS systems. Tracking technologies can be easily categorized into the following groups: 1) mechanical-based tracking including active robots (DaVinci robots [http://www.intuitivesurgical.com, Aug. 2, 2010]) and passive-encoded mechanical arms (Faro mechanical arms [http://products.faro.com/product-overview, Aug. 2, 2010]), 2) optical-based tracking (NDI OptoTrak [http://www.ndigital.com, Aug. 2, 2010], MicronTracker [http://www.clarontech.com, Aug. 2, 2010]), 3) acoustic-based tracking, and 4) electromagnetic (EM)-based tracking (Ascension Technology [http://www.ascension-tech.com, Aug. 2, 2010]).

Ultrasound is one useful imaging modality for image-guided interventions including ablative procedures, biopsy, radiation therapy, and surgery. In the literature and in research labs, ultrasound-guided intervention research is performed by integrating a tracking system (either optical or EM methods) with an ultrasound (US) imaging system to, for example, track and guide liver ablations, or in external beam radiation therapy [E. M. Boctor, M. DeOliviera, M. Choti, R. Ghanem, R. H. Taylor, G. Hager, G. Fichtinger, “Ultrasound Monitoring of Tissue Ablation via Deformation Model and Shape Priors”, International Conference on Medical Image Computing and Computer-Assisted Intervention, MICCAI 2006; H. Rivaz, I. Fleming, L. Assumpcao, G. Fichtinger, U. Hamper, M. Choti, G. Hager, and E. Boctor, “Ablation monitoring with elastography: 2D in-vivo and 3D ex-vivo studies”, International Conference on Medical Image Computing and Computer-Assisted Intervention, MICCAI 2008; H. Rivaz, P. Foroughi, I. Fleming, R. Zellars, E. Boctor, and G. Hager, “Tracked Regularized Ultrasound Elastography for Targeting Breast Radiotherapy”, Medical Image Computing and Computer Assisted Intervention (MICCAI) 2009]. On the commercial side, Siemens and GE Ultrasound Medical Systems recently launched a new interventional system, where an EM tracking device is integrated into high-end cart-based systems. Small EM sensors are integrated into the ultrasound probe, and similar sensors are attached and fixed to the intervention tool of interest.

Limitations of the current approach on both the research and commercial sides can be attributed to the available tracking technologies and to the feasibility of integrating these systems and using them in clinical environments. For example, mechanical-based trackers are considered expensive and intrusive solutions, i.e. they require large space and limit user motion. Acoustic tracking does not provide sufficient navigation accuracy, leaving optical and EM tracking as the most successful and commercially available tracking technologies. However, both technologies require intrusive setups with a base camera (in case of optical tracking methods) or a reference EM transmitter (in case of EM methods). Additionally, optical rigid-body or EM sensors have to be attached to the imager and all needed tools, hence require offline calibration and sterilization steps. Furthermore, none of these systems natively assist multi-modality fusion (registration e.g. between pre-operative CT/MRI plans and intra-operative ultrasound), and do not contribute to direct or augmented visualization either. Thus there remains a need for improved imaging devices for use in image-guided surgery.

### SUMMARY

An augmentation device for an imaging system according to an embodiment of the current invention has a bracket structured to be attachable to an imaging component, and a projector attached to the bracket. The projector is arranged and configured to project an image onto a surface in conjunction with imaging by the imaging system.

A system for image-guided surgery according to an embodiment of the current invention has an imaging system, and a projector configured to project an image or pattern onto a region of interest during imaging by the imaging system.

A capsule imaging device according to an embodiment of the current invention has an imaging system, and a local sensor system. The local sensor system provides information to reconstruct positions of the capsule endoscope free from external monitoring equipment.

### BRIEF DESCRIPTION OF THE DRAWINGS

Further objectives and advantages will become apparent from a consideration of the description, drawings, and examples.

FIG. 1 shows an embodiment of an augmentation device for an imaging system according to an embodiment of the current invention.

FIG. 2 is a schematic illustration of the augmentation device of FIG. 1 in which the bracket is not shown.

FIGS. 3A-3I are schematic illustrations of augmentation devices and imaging systems according to some embodiments of the current invention.

FIG. 4 is a schematic illustration of a system for (MRI-)image-guided surgery according to an embodiment of the current invention.

FIG. 5 is a schematic illustration of a capsule imaging device according to an embodiment of the current invention.

FIGS. 6A and 6B are schematic illustrations of an augmentation device for a handheld imaging system according to an embodiment including a switchable semi-transparent screen for projection purposes.

FIG. 7 is a schematic illustration of an augmentation device for a handheld imaging system according to an embodiment including a laser-based system for photoacoustic imaging (utilizing both tissue- and airborne laser and ultrasound waves) for needle tracking and improved imaging quality in some applications.

FIGS. 8A and 8B are schematic illustrations of one possible approach for needle guidance, using projected guidance information overlaid directly onto the imaged surface, with an intuitive dynamic symbol scheme for position/orientation correction support.

FIG. 9 shows the appearance of a needle touching a surface in a structured light system for an example according to an embodiment of the current application.

FIG. 10 shows surface registration results using CPD on points acquired from CT and a ToF camera for an example according to an embodiment of the current application.

FIG. 11 shows a comparison of SNR and CNR values that show a large improvement in quality and reliability of strain calculation when the RF pairs are selected using our automatic frame selection method for an example according to an embodiment of the current application.

FIG. 12 shows a breast phantom imaged with a three-color sine wave pattern; right the corresponding 3D reconstruction for an example according to an embodiment of the current application.

FIG. 13 shows laparoscopic partial nephrectomy guided by US elasticity imaging for an example according to an embodiment of the current application. Left: System concept and overview. Right: Augmented visualization.

FIG. 14 shows laparoscopic partial nephrectomy guided by US probe placed outside the body for an example according to an embodiment of the current application.

FIG. 15 shows an example of a photoacoustic-based registration method according to an embodiment of the current application. The pulsed laser projector initiates a pattern that can generate PA signals in the US space. Hence, fusion of both US and Camera spaces can be easily established using point-to-point real-time registration method.

FIG. 16 shows ground truth (left image) reconstructed by the complete projection data according to an embodiment of the current application. The middle one is reconstructed using the truncated sonogram with 200 channels trimmed from both sides. The right one is constructed using the truncated data and the extracted trust region (Rectangle support).

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

Some embodiments of this invention describes IGI-(image-guided interventions)-enabling “platform technology” going beyond the current paradigm of relatively narrow image-guidance and tracking. It simultaneously aims to overcome limitations of tracking, registration, visualization, and guidance; specifically using and integrating techniques e.g. related to needle identification and tracking using 3D computer vision, structured light, and photoacoustic effects; multi-modality registration with novel combinations of orthogonal imaging modalities; and imaging device tracking using local sensing approaches; among others.

The current invention covers a wide range of different embodiments, sharing a tightly integrated common core of components and methods used for general imaging, projection, vision, and local sensing.

Some embodiments of the current invention are directed to combining a group of complementary technologies to provide a local sensing approach that can provide enabling technology for the tracking of medical imaging devices, for example, with the potential to significantly reduce errors and increase positive patient outcomes. This approach can provide a platform technology for the tracking of ultrasound probes and other imaging devices, intervention guidance, and information visualization according to some embodiments of the current invention. By combining ultrasound imaging with image analysis algorithms, probe-mounted camera and projection units, and very low-cost, independent optical-inertial sensors, according to some embodiments of the current invention, it is possible to reconstruct the position and trajectory of the device and possible tools or other objects by incrementally tracking their current motion.

Some embodiments of the current invention allow the segmentation, tracking, and guidance of needles and other tools (using visual, ultrasound, and possibly other imaging and localization modalities), allowing for example the integration with the above-mentioned probe tracking capabilities into a complete tracked, image-guided intervention system.

The same set of sensors can enable interactive, in-place visualization using additional projection components. This visualization can include current or pre-operative imaging data or fused displays thereof, but also navigation information such as guidance overlays.

The same projection components can help in surface acquisition and multi-modality registration, capable of reliable and rapid fusion with pre-operative plans, in diverse systems such as handheld ultrasound probes, MRI/CT/C-arm imaging systems, wireless capsule endoscopy, and conventional endoscopic procedures, for example.

Such devices can allow imaging procedures with improved sensitivity and specificity as compared to the current state of the art. This can open up several possible application scenarios that previously required harmful X-ray/CT or expensive MRI imaging, and/or external tracking, and/or expensive, imprecise, time-consuming, or impractical hardware setups, or that were simply afflicted with an inherent lack of precision and guarantee of success, such as:
diagnostic imaging in cancer therapy, prenatal imaging etc.: can allow the generation of freehand three-dimensional ultrasound volumes without the need for external tracking,
biopsies, RF/HIFU ablations etc.: can allow 2D- or 3D-ultrasound-based needle guidance without external tracking,
brachytherapy: can allow 3D-ultrasound acquisition and needle guidance for precise brachytherapy seed placement,
cone-beam CT reconstruction: can enable high-quality C-arm CT reconstructions with reduced radiation dose and focused field of view,
gastroenterology: can perform localization and trajectory reconstruction for wireless capsule endoscopes over extended periods of time, and
other applications relying on tracked imaging and tracked tools.

Some embodiments of the current invention can provide several advantages over existing technologies, such as combinations of:
single-plane US-to-CT/MRI registration—no need for tedious acquisition of US volumes,
low-cost tracking no optical or electro-magnetic (EM) tracking sensors on handheld imaging probes, tools, or needles, and no calibrations necessary,
in-place visualization—guidance information and imaging data is not displayed on a remote screen, but shown projected on the region of interest or over it onto a screen,
local, compact, and non intrusive solution—ideal tracking system for hand-held and compact ultrasound systems that are primarily used in intervention and point-of-care clinical suites, but also for general needle/tool tracking under visual tracking in other interventional settings,
improved quality of cone-beam CT—truncation artifacts are minimized.
improved tracking and multi-modality imaging for capsule endoscopes—enables localization and diagnosis of suspicious findings,
improved registration of percutaneous ultrasound and endoscopic video, using pulsed-laser photoacoustic imaging.

For example, some embodiments of the current invention are directed to devices and methods for the tracking of ultrasound probes and other imaging devices. By combining ultrasound imaging with image analysis algorithms, probe-mounted cameras, and very low-cost, independent optical-inertial sensors, it is possible to reconstruct the position and trajectory of the device and possible tools or other objects by incrementally tracking their current motion according to an embodiment of the current invention. This can provide several possible application scenarios that previously required expensive, imprecise, or impractical hardware setups. Examples can include the generation of freehand three-dimensional ultrasound volumes without the need for external tracking, 3D ultrasound-based needle guidance without external tracking, improved multi-modal registration, simplified image overlay, or localization and trajectory reconstruction for wireless capsule endoscopes over extended periods of time, for example.

The same set of sensors can enable interactive, in-place visualization using additional projection components according to some embodiments of the current invention.

Current sonographic procedures mostly use handheld 2D ultrasound (US) probes that return planar image slices through the scanned 3D volume (the “region of interest”/ROI). In this case, in order to gain sufficient understanding of the clinical situation, the sonographer needs to scan the ROI from many different positions and angles and mentally assemble a representation of the underlying 3D geometry. Providing a computer system with the sequence of 2D images together with the transformations between successive images (“path”) can serve to algorithmically perform this reconstruction of a complete 3D US volume. While this path can be provided by conventional optical, EM etc. tracking devices, a solution of substantially lower cost would hugely increase the use of 3D ultrasound.

For percutaneous interventions requiring needle guidance, prediction of the needle trajectory is currently based on tracking with sensors attached to the distal (external) needle end and on mental extrapolation of the trajectory, relying on the operator's experience. An integrated system with 3D ultrasound, needle tracking, needle trajectory prediction and interactive user guidance would be highly beneficial.

For wireless capsule endoscopes, difficult tracking during the oesophago-gastro-intestinal passage is a major obstacle to exactly localized diagnoses. Without knowledge about the position and orientation of the capsule, it is impossible to pinpoint and quickly target, tumors and other lesions for therapy. Furthermore, diagnostic capabilities of current wireless capsule endoscopes are limited. With a low-cost localization and lumen reconstruction system that does not rely on external assembly components, and with integrated photoacoustic sensing, much improved outpatient diagnoses can be enabled.

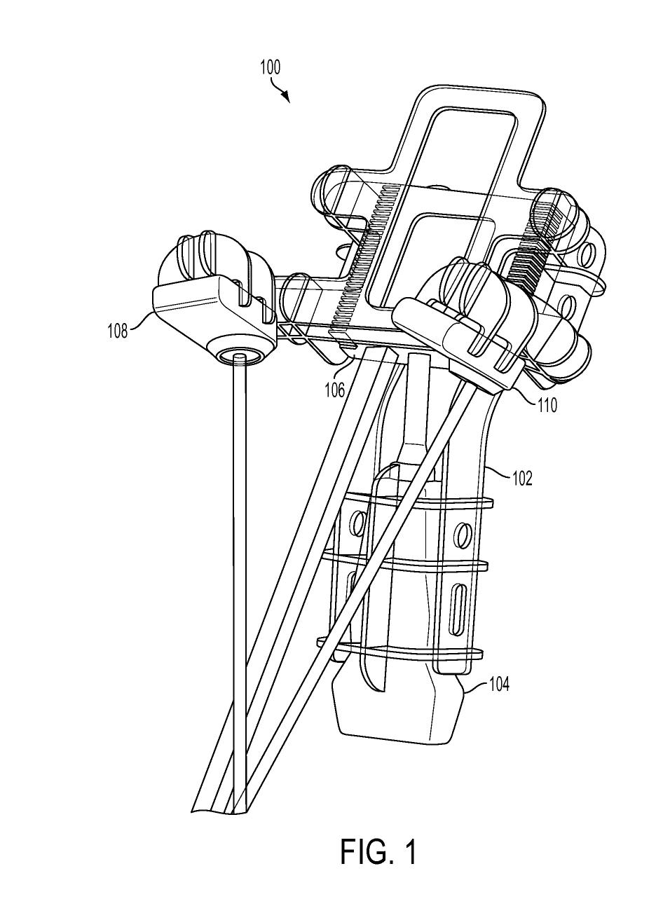

FIG. 1 is an illustration of an embodiment of an augmentation device 100 for an imaging system according to an embodiment of the current invention. The augmentation device 100 includes a bracket 102 that is structured to be attachable to an imaging component 104 of the imaging system. In the example of FIG. 1, the imaging component 104 is an ultrasound probe and the bracket 102 is structured to be attached to a probe handle of the ultrasound probe. However, the broad concepts of the current invention are not limited to only this example. The bracket 102 can be structured to be attachable to other handheld instruments for image-guided surgery, such as surgical orthopedic power tools or stand-alone handheld brackets, for example. In other embodiments, the bracket 102 can be structured to be attachable to the C-arm of an X-ray system or an MRI system, for example.

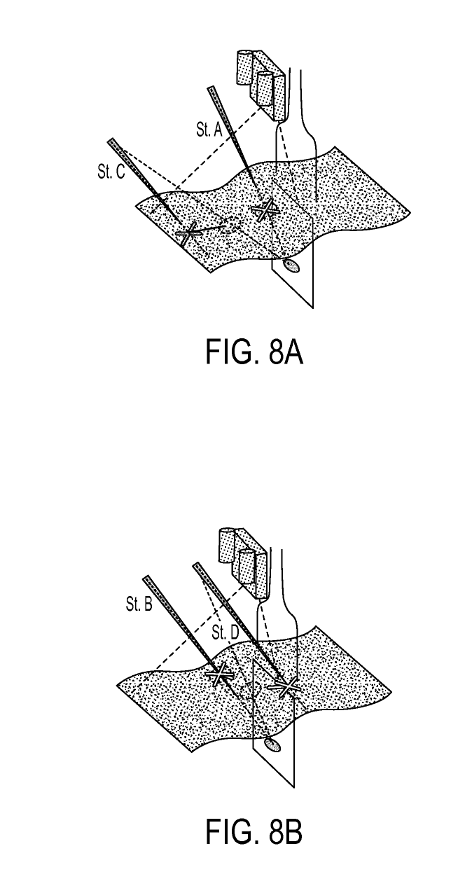

The augmentation device 100 also includes a projector 106 attached to the bracket 102. The projector 106 is arranged and configured to project an image onto a surface in conjunction with imaging by the imaging component 104. The projector 106 can be at least one of a visible light imaging projector, a laser imaging projector, a pulsed laser, or a projector of a fixed or selectable pattern (using visible, laser, or infrared/ultraviolet light). Depending on the application, the use of different spectral ranges and power intensities enables different capabilities, such as infrared for structured light illumination simultaneous with e.g. visible overlays; ultraviolet for UV-sensitive transparent glass screens (such as MediaGlass, SuperImaging Inc.); or pulsed laser for photoacoustic imaging, for example. A fixed pattern projector can include, for example, a light source arranged to project through a slide, a mask, a reticle, or some other light-patterning structure such that a predetermined pattern is projected onto the region of interest. This can be used, for example, for projecting structured light patterns (such as grids or locally unique patterns) onto the region of interest (U.S. Pat. No. 7,103,212 B2, Hager et al., the entire contents of which is incorporated herein by reference). Another use for such projectors can be the overlay of user guidance information onto the region of interest, such as dynamic needle-insertion-supporting symbols (circles and crosses, cf. FIG. 8). Such a projector can be made to be very compact in some applications. A projector of a selectable pattern can be similar to the fixed pattern device, but with a mechanism to select and/or exchange the light-patterning component. For example, a rotating component could be used in which one of a plurality of predetermined light-patterning sections is moved into the path of light from the light source to be projected onto the region of interest. In other embodiments, said projector(s) can be a stand-alone element of the system, or combined with a subset of other components described in the current invention, i.e. not necessarily integrated in one bracket or holder with another imaging device. In some embodiments, the projector(s) may be synchronized with the camera(s), imaging unit, and/or switchable film screens.

The augmentation device 100 can also include at least one of a camera 108 attached to the bracket 102. In some embodiments, a second camera 110 can also be attached to the bracket 102, either with or without the projector, to provide stereo vision, for example. The camera can be at least one of a visible-light camera, an infra-red camera, or a time-of-flight camera in some embodiments of the current invention. The camera(s) can be stand-alone or integrated with one or more projection units in one device as well, depending on the application. They may have to be synchronized with the projector(s) and/or switchable film glass screens as well.

Additional cameras and/or projectors could be provided—either physically attached to the main device, some other component, or free-standing—without departing from the general concepts of the current invention.

The camera 108 and/or 110 can be arranged to observe a surface region close to the and during operation of the imaging component 104. In the embodiment of FIG. 1, the two cameras 108 and 110 can be arranged and configured for stereo observation of the region of interest. Alternatively, one of the cameras 108 and 110, or an additional camera, or two, or more, can be arranged to track the user face location during visualization to provide information regarding a viewing position of the user. This can permit, for example, the projection of information onto the region of interest in such a way that it takes into account the position of the viewer, e.g. to address the parallax problem.

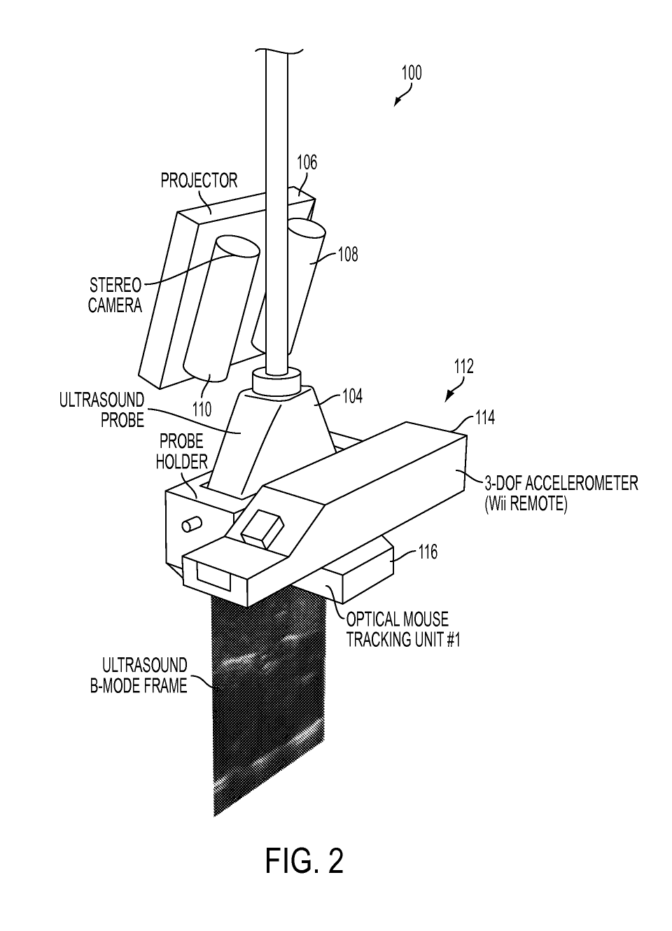

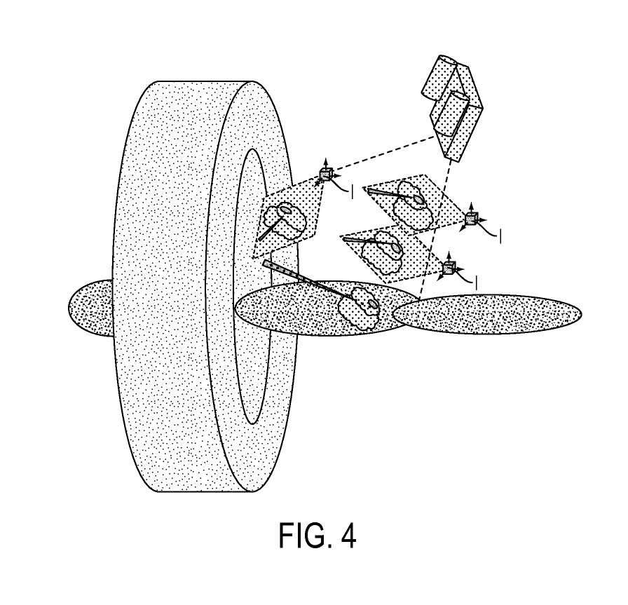

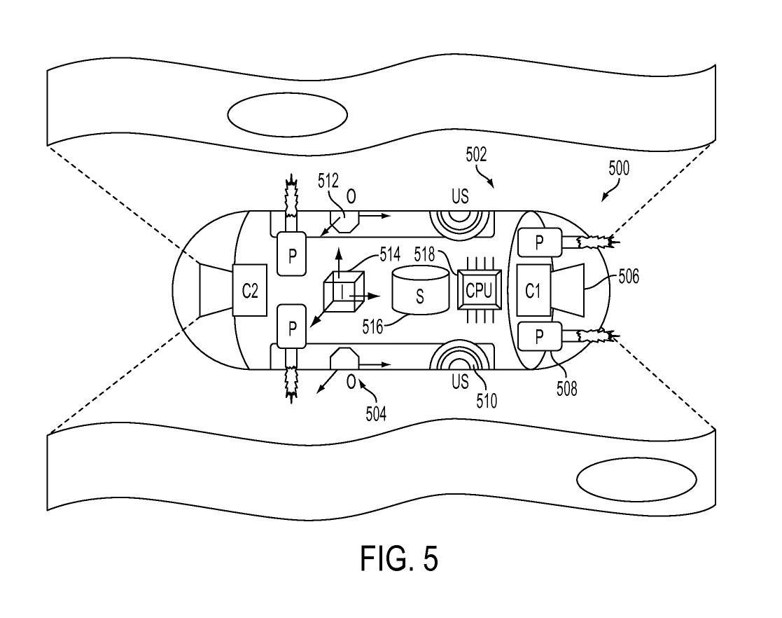

FIG. 2 is a schematic illustration of the augmentation device 100 of FIG. 1 in which the bracket 102 is not shown for clarity. FIG. 2 illustrates further optional local sensing components that can be included in the augmentation device 100 according to some embodiments of the current invention. For example, the augmentation device 100 can include a local sensor system 112 attached to the bracket 102. The local sensor system 112 can be part of a conventional tracking system, such as an EM tracking system, for example. Alternatively, the local sensor system 112 can provide position and/or orientation information of the imaging component 104 to permit tracking of the imaging component 104 while in use without the need for external reference frames such as with conventional optical or EM tracking systems. Such local sensor systems can also help in the tracking (e.g. determining the orientation) of handheld screens (FIG. 4) or capsule endoscopes (FIG. 5), not just of imaging components. In some embodiments, the local sensor system 112 can include at least one of an optical, inertial, or capacitive sensor, for example. In some embodiments, the local sensor system 112 includes an inertial sensor component 114 which can include one or more gyroscopes and/or, linear accelerometers, for example. In one embodiment, the local sensor system 112 has a three-axis gyro system that provides rotation information about three orthogonal axes of rotation. The three-axis gyro system can be a micro-electromechanical system (MEMS) three-axis gyro system, for example. The local sensor system 112 can alternatively, or in addition, include one or more linear accelerometers that provide acceleration information along one or more orthogonal axes in an embodiment of the current invention. The linear accelerometers can be, for example, MEMS accelerometers.

In addition to, or instead of the inertial sensor component 114, the local sensor system 112 can include an optical sensor system 116 arranged to detect motion of the imaging component 104 with respect to a surface. The optical sensor system 116 can be similar to the sensor system of a conventional optical mouse (using visible, IR, or laser light), for example. However, in other embodiments, the optical sensor system 116 can be optimized or otherwise customized for the particular application. This may include the use of (potentially stereo) cameras with specialized feature and device tracking algorithms (such as scale-invariant feature transform/SIFT and simultaneous localization and mapping/SLAM, respectively) to track the device, various surface features, or surface region patches over time, supporting a variety of capabilities such as trajectory reconstruction or stereo surface reconstruction.

In addition to, or instead of the inertial sensor component 114, the local sensor system 112 can include a local ultrasound sensor system to make use of the airborne photoacoustic effect. In this embodiment, one or more pulsed laser projectors direct laser energy towards the patient tissue surface, the surrounding area, or both, and airborne ultrasound receivers placed around the probe itself help to detect and localize potential objects such as tools or needles in the immediate vicinity of the device.

In some embodiments, the projector 106 can be arranged to project an image onto a local environment adjacent to the imaging component 104. For example, the projector 106 can be adapted to project a pattern onto a surface in view of the cameras 108 and 110 to facilitate stereo object recognition and tracking of objects in view of the cameras. For example, structured light can be projected onto the skin or an organ of a patient according to some embodiments of the current invention. According to some embodiments, the projector 106 can be configured to project an image that is based on ultrasound imaging data obtained from the ultrasound imaging device. In some embodiments, the projector 106 can be configured to project an image based on imaging data obtained from an x-ray computed tomography imaging device or a magnetic resonance imaging device, for example. Additionally, preoperative data or real-time guidance information could also be projected by the projector 106.

The augmentation device 100 can also include a communication system that is in communication with at least one of the local sensor system 112, camera 108, camera 110 or projector 106 according to some embodiments of the current invention. The communication system can be a wireless communication system according to some embodiments, such as, but not limited to, a Bluetooth wireless communication system.

Although FIGS. 1 and 2 illustrate the imaging system as an ultrasound imaging system and that the bracket 102 is structured to be attached to an ultrasound probe handle 104, the broad concepts of the current invention are not limited to this example. The bracket can be structured to be attachable to other imaging systems, such as, but not limited to, x-ray and magnetic resonance imaging systems, for example.

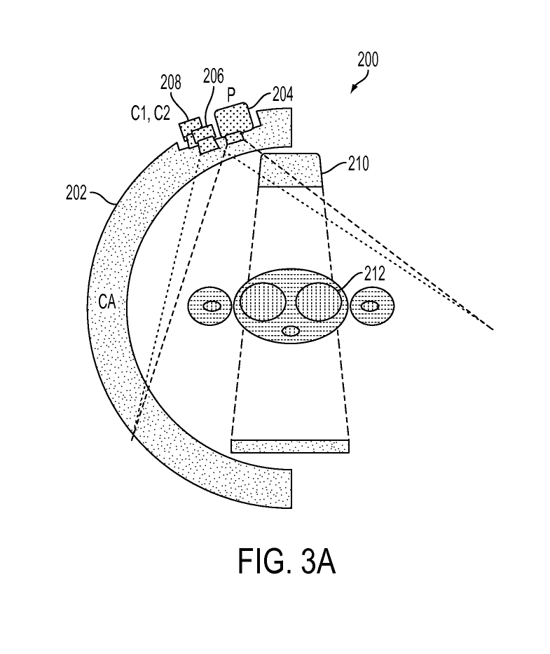

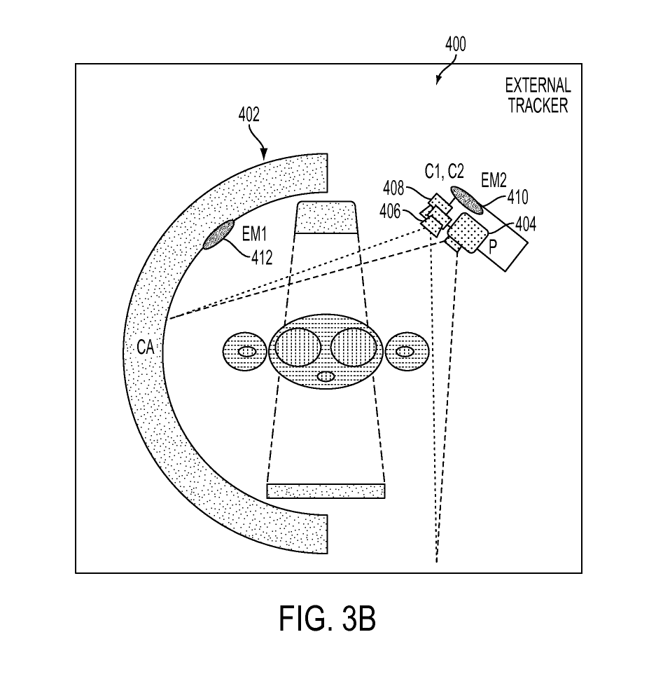

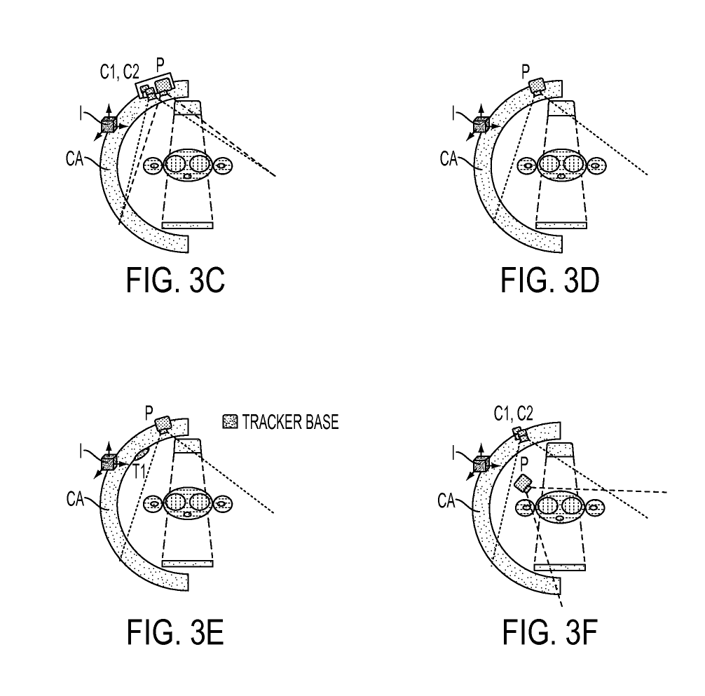

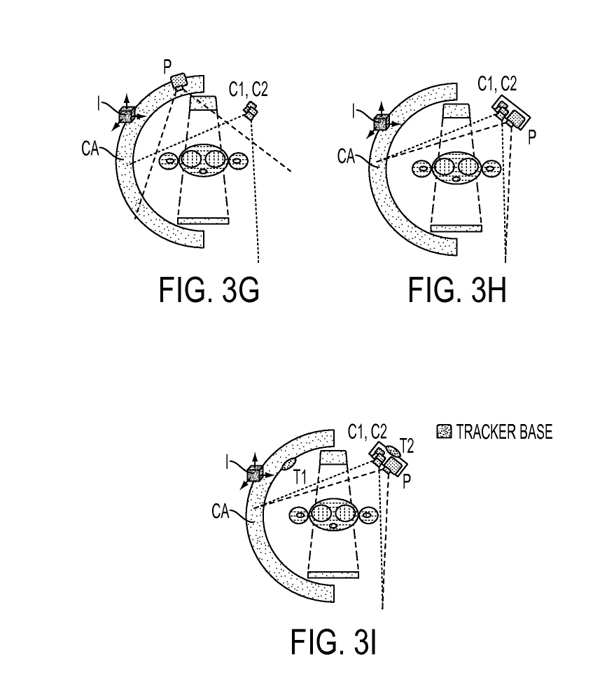

FIG. 3A is a schematic illustration of an augmentation device 200 attached to the C-arm 202 of an x-ray imaging system. In this example, the augmentation device 200 is illustrated as having a projector 204, a first camera 206 and a second camera 208. Conventional and/or local sensor systems can also be optionally included in the augmentation device 200, improving the localization of single C-arm X-ray images by enhancing C-arm angular encoder resolution and estimation robustness against structural deformation.

In operation, the x-ray source 210 typically projects an x-ray beam that is not wide enough to encompass the patient's body completely, resulting in severe truncation artifacts in the reconstruction of so-called cone beam CT (CBCT) image data. The camera 206 and/or camera 208 can provide information on the amount of extension of the patient beyond the beam width. This information can be gathered for each angle as the C-arm 202 is rotated around the patient 212 and be incorporated into the processing of the CBCT image to at least partially compensate for the limited beam width and reduce truncation artifacts [Ismail-2011]. In addition, conventional and/or local sensors can provide accurate data of the precise angle of illumination by the x-ray source, for example (more precise than potential C-arm encoders themselves, and potentially less susceptible to arm deformation under varying orientations). Other uses of the camera-projection combination units are surface-supported multi-modality registration, or visual needle or tool tracking, or guidance information overlay. One can see that the embodiment of FIG. 3A is very similar to the arrangement, of an augmentation device for an MRI system.

FIG. 3B is a schematic illustration of a system for image-guided surgery 400 according to some embodiments of the current invention. The system for image-guided surgery 400 includes an imaging system 402, and a projector 404 configured to project an image onto a region of interest during imaging by the imaging system 402. The projector 404 can be arranged proximate the imaging system 402, as illustrated, or it could be attached to or integrated with the imaging system. In this case, the imaging system 402 is illustrated schematically as an x-ray imaging system. However, the invention is not limited to this particular example. As in the previous embodiments, the imaging system could also be an ultrasound imaging system or a magnetic resonance imaging system, for example. The projector 404 can be at least one of a white light imaging projector, a laser light imaging projector, a pulsed laser, or a projector of a fixed or selectable pattern, for example.

The system for image-guided surgery 400 can also include a camera 406 arranged to capture an image of a region of interest during imaging by the imaging system. A second camera 408 could also be included in some embodiments of the current invention. A third, fourth or even more cameras could also be included in some embodiments. The region of interest being observed by the imaging system 402 can be substantially the same as the region of interest being observed with the camera 406 and/or camera 408. The cameras 406 and 408 can be at least one of a visible-light camera, an infra-red camera or a time-of-flight camera, for example. Each of the cameras 406, 408, etc. can be arranged proximate the imaging system 402 or attached to or integrated with the imaging system 402.

The system for image-guided surgery 400 can also include one or more sensor systems, such as sensor systems 410 and 412, for example. In this example, the sensor systems 410 and 412 are part of a conventional EM sensor system. However, other conventional sensor systems such as optical tracking systems could be used instead of or in addition to the EM sensor systems illustrated. Alternatively, or in addition, one or more local sensor systems such as local sensor system 112 could also be included instead of sensor systems 410 and/or 412. The sensor systems 410 and/or 412 could be attached to any one of the imaging system 402, the projector 404, camera 406 or camera 408, for example. Each of the projector 404 and cameras 406 and 408 could be grouped together or separate and could be attached to or made integral with the imaging system 402, or arranged proximate the imaging system 402, for example.

FIG. 4 illustrates one possible use of a camera/projection combination unit in conjunction with a medical imaging device such as MRI or CT. Image-guided interventions based on these modalities suffer from registration difficulties arising from the fact that in-place interventions are awkward or impossible due to space constraints within the imaging device bores, among other reasons. Therefore, a multi-modality image registration system supporting the interactive overlay of potentially fused pre- and intra-operative image data could support or enable e.g. needle-based percutaneous interventions with massively reduced imaging requirements in terms of duration, radiation exposure, cost etc. A camera/projection unit outside the main imaging system could track the patient, reconstruct the body surface using e.g. structured light and stereo reconstruction, and register and track needles and other tools relative to it. Furthermore, handheld units comprising switchable film glass screens could be tracked optically and used as interactive overlay projection surfaces. The tracking accuracy for such screens could be improved by attaching (at least inertial) local sensor systems to said screens, allowing better orientation estimation that using visual clues alone. The screens need not impede the (potentially structured-light-supported) reconstruction of the underlying patient surface, nor block the user's view of that surface, as they can be rapidly switched (up to hundreds of times per second) alternating between a transparent mode to allow pattern and guidance information projection onto the surface, and an opaque mode to block and display other user-targeted data, e.g. in a tracked 3D data visualization fashion.

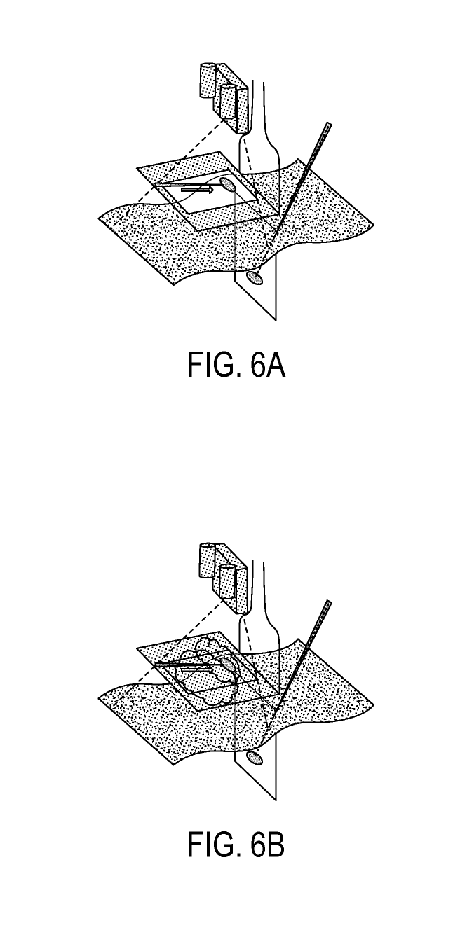

Such switchable film glass screens can also be attached to handheld imaging devices such as ultrasound probes and the afore-mentioned brackets as in FIG. 6. This way, imaging and/or guidance data can be displayed on a handheld screen—in opaque mode—directly adjacent to imaging devices in the region of interest, instead of on a remote monitor screen. Furthermore—in transparent mode—structured light projection and/or surface reconstruction are not impeded by the screen. In both cases the data is projected onto or through the switchable screen using the afore-mentioned projection units, allowing a more compact handheld design (e.g., U.S. Pat. No. 6,599,247 B1, Stetten et al.) or even remote projection. Furthermore, these screens (handheld or bracket-mounted) can also be realized using e.g. UV-sensitive/fluorescent glass, requiring a (potentially multi-spectral for color reproduction) UV projector to create bright images on the screen, but making active control of screen mode switching unnecessary. In the latter case, overlay data projection onto the screen and structured light projection onto the patient surface can be run in parallel, provided the structured light uses a frequency unimpeded by the glass.

FIG. 5 is a schematic illustration of a capsule imaging device 500 according to an embodiment of the current invention. The capsule imaging device 500 includes an imaging system 502 and a local sensor system 504. The local sensor system 504 provides information to reconstruct positions of the capsule imaging device 500 free from external monitoring equipment. The imaging system 502 can be an optical imaging system according to some embodiments of the current invention. In other embodiments, the imaging system 502 can be, or can include, an ultrasound imaging system. The ultrasound imaging system can include, for example a pulsed laser and an ultrasound receiver configured to detect ultrasound signals in response to pulses from said pulsed laser interacting with material in regions of interest. Either the pulsed laser or the ultrasound receivers may be arranged independently outside the capsule, e.g. outside the body, thus allowing higher energy input or higher sensitivity.

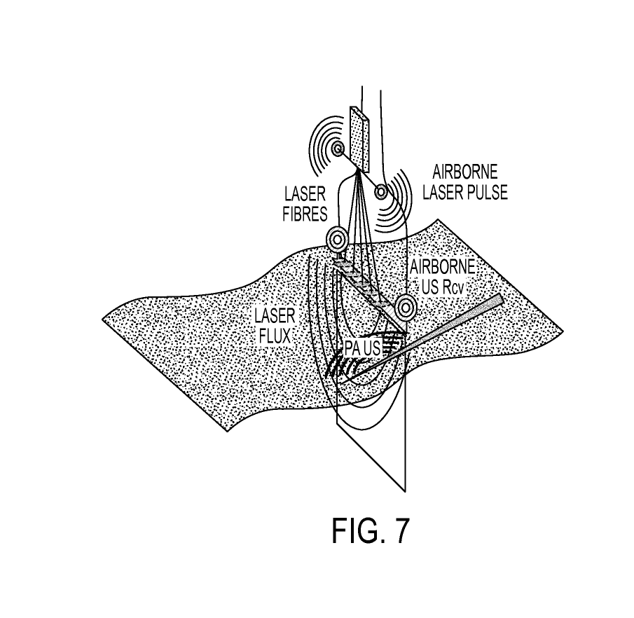

FIG. 7 describes a possible extension to the augmentation device (“bracket”) described for handheld imaging devices, comprising one or more pulsed lasers as projection units that are directed through fibers towards the patient surface, exciting tissue-borne photoacoustic effects, and towards the sides of the imaging device, emitting the laser pulse into the environment, allowing airborne photoacoustic imaging. For the latter, the handheld imaging device and/or the augmentation device comprise ultrasound receivers around the device itself, pointing into the environment. Both photoacoustic channels can be used e.g. to enable in-body and out-of-body tool tracking or out-of-plane needle detection and tracking, improving both detectability and visibility of tools/needles under various circumstances.

In endoscopic systems the photoacoustic effect can be used together with its structured-light aspect for registration between endoscopic video and ultrasound. By emitting pulsed laser patterns from a projection unit in an endoscopic setup, a unique pattern of light incidence locations is generated on the endoscope-facing surface side of observed organs. One or more camera units next to the projection unit in the endoscopic device observe the pattern, potentially reconstructing its three-dimensional shape on the organ surface. At the same time, a distant ultrasound imaging device on the opposite side of the organ under observation receives the resulting photoacoustic wave patterns and is able to reconstruct and localize their origins, corresponding to the pulsed-laser incidence locations. This “rear-projection” scheme allows simple registration between both sides—endoscope and ultrasound—of the system.

FIG. 8 outlines one possible approach to display needle guidance information to the user by means of direct projection onto the surface in the region of interest in a parallax-independent fashion, so the user position is not relevant to the method's success (the same method can be applied to projection e.g. onto a device-affixed screen as described above, or onto handheld screens). Using e.g. a combination of moving, potentially color/size/thickness/etc.-coded circles and crosses, the five degrees of freedom governing a needle insertion (two each for insertion point location and needle orientation, and one for insertion depth and/or target distance) can be intuitively displayed to the user. In one possible implementation, the position and color of a projected circle on the surface indicate the intersection of the line between the current needle position and the target location with the patient surface, and said intersection point's distance from a planned insertion point. The position, color, and size of a projected cross can encode the current orientation of the needle with respect to the correct orientation towards the target location, as well as the needle's distance from the target. The orientation deviation is also indicated by an arrow pointing towards the proper position/orientation configuration. In another implementation, guidance information necessary to adjust the needle orientation can be projected as a virtual shadow onto the surface next to the needle insertion point, prompting the user to minimize the shadow length to properly orient the needle for insertion.

While the above-mentioned user guidance display is independent of the user viewing direction, several other information displays (such as some variations on the image-guided intervention system shown in FIG. 4) may benefit from knowledge about the location of the user's eyes relative to the imaging device, the augmentation device, another handheld camera/projection unit, and/or projection screens or the patient surface. Such information can be gathered using one or more optical (e.g. visible- or infrared-light) cameras pointing away from the imaging region of interest towards regions of space where the user face may be expected (such as upwards from a handheld ultrasound imaging device) combined with face-detection capabilities to determine the user's eye location, for example.

### EXAMPLES

The following provides some examples according to some embodiments of the current invention. These examples are provided to facilitate a description of some of the concepts of the invention and are not intended to limit the broad concepts of the invention.

The local sensor system can include inertial sensors 506, such as a three-axis gyro system, for example. For example, the local sensor system 504 can include a three-axis MEMS gyro system. In some embodiments, the local sensor system 504 can include optical position sensors 508, 510 to detect motion of the capsule imaging device 500. The local sensor system 504 can permit the capsule imaging device 500 to record position information along with imaging data to facilitate registering image data with specific portions of a patient's anatomy after recovery of the capsule imaging device 500, for example.

Some embodiments of the current invention can provide an augmentation of existing devices which comprises a combination of different sensors: an inertial measurement unit based on a 3-axis accelerometer; one or two optical displacement tracking units (OTUs) for lateral surface displacement measurement; one, two or more optical video cameras; and a (possibly handheld and/or linear) ultrasound (US) probe, for example. The latter may be replaced or accompanied by a photoacoustic (PA) arrangement, i.e. one or more active lasers, a photoacoustically active extension, and possibly one or more separate US receiver arrays. Furthermore, an embodiment of the current invention may include a miniature projection device capable of projecting at least two distinct features.

These sensors (or a combination thereof) may be mounted, e.g. on a common bracket or holder, onto the handheld US probe, with the OTUs pointing towards and close to the scanning surface (if more than one, then preferably at opposite sides of the US array), the cameras mounted (e.g., in a stereo arrangement) so they can capture the environment of the scanning area, possible needles or tools, and/or the operating room environment, and the accelerometer in a basically arbitrary but fixed location on the common holder. In a particular embodiment, the projection device may be pointing mainly onto the scanning surface. In another particular embodiment, one PA laser may point towards the PA extension, while the same or another laser may point outwards, with US receiver arrays suitably arranged to capture possible reflected US echos. Different combinations of the mentioned sensors are possible.

For particular applications and/or embodiments, an interstitial needle or other tool may be used. The needle or tool may have markers attached for better optical visibility outside the patient body. Furthermore, the needle or tool may be optimized for good ultrasound visibility if they are supposed to be inserted into the body. In particular embodiments the needle or tool may be combined with inertial tracking components (i.e. accelerometers).

For particular applications and/or embodiments, additional markers may optionally be used for the definition of registration or reference positions on the patient body surface. These may be optically distinct spots or arrangements of geometrical features designed for visibility and optimized optical feature extraction.

For particular applications and/or embodiments, the device to be augmented by the proposed invention may be a handheld US probe; for others it may be a wireless capsule endoscope (WCE); and other devices are possible for suitably defined applications, where said applications may benefit from the added tracking and navigational capabilities of the proposed invention.

### Software Components:

In one embodiment (handheld US probe tracking), an embodiment of the invention includes a software system for opto-inertial probe tracking (OIT). The OTUs generate local translation data across the scan surface (e.g. skin or intestinal wall), while accelerometers and/or gyroscopes provide absolute orientation and/or rotation motion data. Their streams of local data are combined over time to reconstruct an n-DoF probe trajectory with n=2 . . . 6, depending on the actual OIC sensor combination and the current pose/motion of the probe.

In general, the current pose Q(t)=(P(t), R(t)) can be computed incrementally with

$$P ( t ) = P ( 0 ) + {\sum }_{i = 0}^{t - 1} R ( i ) Δ p ( i )$$

where the R(i) are the orientations directly sampled from the accelerometers and/or incrementally tracked from relative displacements between the OTUs (if more than one) at time i, and Δp(i) are the lateral displacements at time i as measured by the OTUs. P(0) is an arbitrarily chosen initial reference position.

In one embodiment (handheld US probe tracking), a software system for speckle-based probe tracking is included. An (ultrasound-image-based) speckle decorrelation analysis (SDA) algorithm provides very high-precision 1-DoF translation (distance) information for single ultrasound image patch pairs by decorrelation, and 6-DoF information for the complete ultrasound image when combined with planar 2D-2D registration techniques. Suitable image patch pairs are preselected by means of FDS (fully developed speckle) detection. Precision of distance estimation is improved by basing the statistics on a larger set of input pairs.

Both approaches (opto-inertial tracking and SDA) may be combined to achieve greater efficiency and/or robustness. This can be achieved by dropping the FDS detection step in the SDA and instead relying on opto-inertial tracking to constrain the set of patch pairs to be considered, thus implicitly increasing the ratio of suitable FDS patches without explicit FDS classification.

Another approach can be the integration of opto-inertial tracking information into a maximum-a-posteriori (MAP) displacement estimation. In yet another approach, sensor data fusion between OIT and SDA can be performed using a Kalman filter.

In one embodiment (handheld US probe tracking), a software system for camera-based probe tracking and needle and/or tool tracking and calibration can be included.

The holder-mounted camera(s) can detect and segment e.g. a needle in the vicinity of the system. By detecting two points P₁ and P₂, with P₁ being the needle insertion point into the patient tissue (or alternatively, the surface intersection point in a water container) and P₂ being the end or another suitably distant point on the needle, and a third point P₁ being the needle intersection point in the US image frame, it is possible to calibrate the camera-US probe system in one step in closed form by following

(P₂−P₁)×(P₁−XPᵢ)=0

with X being the sought calibration matrix linking US frame and the camera(s).

Furthermore, if the above-mentioned calibration condition does not hold at some point in time (detectable by the camera(s)), needle bending can be inferred from a single 2D US image frame and the operator properly notified.

Furthermore, 3D image data registration is also aided by the camera(s) overlooking the patient skin surface. Even under adverse geometrical conditions, three degrees of freedom (tilt, roll, and height) can be constrained using the cameras, facilitating registration of 3D US and e.g. CT or similar modalities by restricting the registration search space (making it faster) or providing initial transformation estimates (making it easier and/or more reliable). This may be facilitated by the application of optical markers onto the patient skin surface, which will also help in the creation of an explicit fixed reference coordinate system for integration of multiple 3D volumes.

Furthermore, the camera(s) provide additional data for pose tracking. In general, this will consist of redundant rotational motion information in addition to opto-inertial tracking. In special cases however, this information could not be recovered from OIT (e.g. yaw motions on a horizontal plane in case of surface tracking loss of one or both optical translation detectors, or tilt motion without translational components around a vertical axis). This information may originate from a general optical-flow-based rotation estimation, or specifically from tracking of specially applied optical markers onto the patient skin surface, which will also help in the creation of an explicit fixed reference coordinate system for integration of multiple 3D volumes.

Furthermore, by detecting and segmenting the extracorporeal parts of a needle, the camera(s) can provide needle translation information. This can serve as input for ultrasound elasticity imaging algorithms to constrain the search space (in direction and magnitude) for the displacement estimation step by tracking the needle and transforming estimated needle motion into expected motion components in the US frame, using the aforementioned calibration matrix X.

Furthermore, the camera(s) can provide dense textured 3D image data of the needle insertion area. This can be used to provide enhanced visualization to the operator, e.g. as a view of the insertion trajectory as projected down along the needle shaft towards the skin surface, using actual needle/patient images.

For particular applications and/or embodiments, integration of a micro-projector unit can provide an additional, real-time, interactive visual user interface e.g. for guidance purposes. Projecting navigation data onto the patient skin in the vicinity of the probe, the operator need not take his eyes away from the intervention site to properly target subsurface regions. Tracking the needle using the aforementioned camera(s), the projected needle entry point (intersection of patient skin surface and extension of the needle shaft) given the current needle position and orientation can be projected using a suitable representation (e.g. a red dot). Furthermore, an optimal needle entry point given the current needle position and orientation can be projected onto the patient skin surface using a suitable representation (e.g. a green dot). These can be positioned in real-time, allowing interactive repositioning of the needle before skin puncture without the need for external tracking.

Different combinations of software components are possible for different applications and/or different hardware embodiments.

For wireless capsule endoscope (WCE) embodiments, using the photoacoustic effect with the photoacoustic (PA) arrangement provides additional tracking information as well as an additional imaging modality.

In environments like the gastrointestinal (GI) tract, wall contact may be lost intermittently. In contact situations, OIT can provide sufficient information to track the WCE over time, while in no-contact ones the PA laser can fire at the PA arrangement to excite an emitted sound wave that is almost perfectly reflected from the surrounding walls and received using a passive US receive array. This can provide wall shape information that can be tracked over time to estimate displacement.

For imaging, the PA laser can fire directly and diffusely at the tissue wall, exciting a PA sound wave emanating from there that is received with the mentioned passive US array and can be used for diagnostic purposes. Ideally, using a combination of the mentioned tracking methods, the diagnostic outcome can be linked to a particular location along the GI tract.

Some embodiments of the current invention can allow reconstructing a 2D ultrasound probe's 6-DoF (“degrees of freedom”) trajectory robustly, without the need for an external tracking device. The same mechanism can be e.g. applied to (wireless) capsule endoscopes as well. This can be achieved by cooperative sets of local sensors that incrementally track a probe's location through its sequence of motions. Some aspects of the current invention can be summarized, as follows.

First, an (ultrasound-image-based) speckle decorrelation analysis (SDA) algorithm provides very high-precision 1-DoF translation (distance) information for image patch pairs by decorrelation, and 6-DoF information for the complete ultrasound image when combined with planar 2D-2D registration techniques. Precision of distance estimation is improved by basing the statistics on a larger set of input pairs. (The parallelized approach with a larger input image set can significantly increase speed and reliability.)

Additionally, or alternatively, instead of using a full transmit/receive ultrasound transceiver (e.g. because of space or energy constraints, as in a wireless capsule endoscope), only an ultrasound receiver can be used according to some embodiments of the current invention. The activation energy in this case comes from an embedded laser. Regular laser discharges excite irregularities in the surrounding tissue and generate photoacoustic impulses that can be picked up with the receiver. This can help to track surfaces and subsurface features using ultrasound and thus provide additional information for probe localization.

Second, a component, bracket, or holder housing a set of optical, inertial, and/or capacitive (OIC) sensors represents an independent source of (ultrasound-image-free) motion information. Optical displacement trackers (e.g. from optical mice or cameras) generate local translation data across the scan surface (e.g. skin or intestinal wall), while accelerometers and/or gyroscopes provide absolute orientation and/or rotation motion data. Capacitive sensors can estimate the distance to tissue when the optical sensors loses surface contact or otherwise suffers tracking loss. Their streams of local data are combined over time to reconstruct an n-DoF probe trajectory with n=2 . . . 6, depending on the actual OIC sensor combination and the current pose/motion of the probe.

Third, two or more optical video cameras are attached to the ultrasound probe, possibly in stereo fashion, at vantage points that let them view the surrounding environment, including any or all of the patient skin surface, possible tools and/or needles, possible additional markers, and parts of the operation room environment. This way, they serve to provide calibration, image data registration support, additional tracking input data, additional input data supporting ultrasound elasticity imaging, needle bending detection input, and/or textured 3D environment model data for enhanced visualization.

In a last step, the information (partly complementary, partly redundant) from all three local sensor sets (OIC, SDA, and optical cameras) serves as input to a filtering or data fusion algorithm. All of the sensors cooperatively augment each others' data: OIC tracking informs the SDA about the direction of motion (which is hard to recover from SDA alone), while SDA provides very-high precision small-scale displacement information. Orientation information is extracted from the OIC sensors, while the SDA provides rotational motion information. Additionally, the optical cameras can support orientation estimation, especially in geometrically degenerate cases where OIC and possibly SDA might fail. This data fusion can be performed using any of a variety of different filtering algorithms, e.g. a Kalman filter (assuming a model of the possible device motion) or a Maximum a posteriori (MAP) estimation (when the sensor measurement distributions for actual device motions can be given). The final 6-DoF trajectory is returned incrementally and can serve as input to a multitude of further processing steps, e.g. 3D-US volume reconstruction algorithms or US-guided needle tracking applications.

Furthermore, by incorporating additional local sensors (like the OIC sensor bracket) beyond using the ultrasound RF data for the speckle decorrelation analysis (SDA), it is possible to simplify algorithmic complexity and improve robustness by dropping the detection of fully developed speckle (FDS) patches before displacement estimation. While this FDS patch detection is traditionally necessary for SDA, using OIC will provide constraints for the selection of valid patches by limiting the space of possible patches, thus increasing robustness e.g. in combination with RANSAC subset selection algorithms.

Finally, a micro-projection device (laser- or image-projection-based) integrated into the ultrasound probe bracket can provide the operator with an interactive, real-time visualization modality, displaying relevant data like needle intersection points, optimal entry points, and other supporting data directly in the intervention location by projecting these onto the patient skin surface near the probe.

The embodiments illustrated and discussed in this specification are intended only to teach those skilled in the art the best way known to the inventors to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Example 1

### Ultrasound-Guided Liver Ablation Therapy

Recent evidence suggests thermal ablation in some cases can achieve results comparable to that of resection. Specifically, a recent randomized clinical trial comparing resection to RFA for small HCC found equivalent long-term outcomes with lower morbidity in the ablation arm [Chen-2006]. Importantly, most studies suggest that efficacy of RFA is highly dependent on the experience and diligence of the treating physician, often associated with a steep learning curve [Poon-2004]. Moreover, the apparent efficacy of open operative RFA over a percutaneous approach reported by some studies suggest that difficulty with targeting and imaging may be contributing factors [Mulier-2005]. Studies of the failure patterns following RFA similarly suggest that limitations in real-time imaging, targeting, monitoring of ablative therapy are likely contributing to increased risk of local recurrence [Mulier-2005].

One of the most useful features of ablative approaches such as RFA is that it can be applied using minimally invasive techniques. Length of hospital stay, costs, and morbidity may be reduced using this technique [Berber-2008]. These benefits add to the appeal of widening the application of local therapy for liver tumors to other tumor types, perhaps in combination with more effective systemic therapies for minimal residual disease. Improvements in the control, size, and speed of tumor destruction with RFA will begin to allow us to reconsider treatment options for such patients with liver tumors as well. However, clinical outcomes data are clear—complete tumor destruction with adequate margins is imperative in order to achieve durable local control and survival benefit, and this should be the goal of any local therapy. Partial, incomplete, or palliative local therapy is rarely indicated. One study even suggested that incomplete destruction with residual disease may in fact be detrimental, stimulating tumor growth of locally residual tumor cells [Koichi-2008]. This concept is often underappreciated when considering tumor ablation, leading to lack of recognition by some of the importance of precise and complete tumor destruction. Improved targeting, monitoring, and documentation of adequate ablation are critical to achieve this goal. Goldberg et al, in the most cited work on this subject [Goldberg-2000], describes an ablative therapy framework in which the key areas in advancing this technology include improving (1) image guidance, (2) intra-operative monitoring, as well as (3) ablation technology itself.

In spite of promising results of ablative therapies, significant technical barriers exist with regard to its efficacy, safety, and applicability to many patients. Specifically, these limitations include: (1) localization/targeting of the tumor and (2) monitoring of the ablation zone.

Targeting Limitations: One common feature of current ablative methodology is the necessity for precise placement of the end-effector tip in specific locations, typically within the volumetric center of the tumor, in order to achieve adequate destruction. The tumor and zone of surrounding normal parenchyma can then be ablated. Tumors are identified by preoperative imaging, primarily CT and MR, and then operatively (or laparoscopically) localized by intra-operative ultrasonography (IOUS). When performed percutaneously, trans-abdominal ultrasonography is most commonly used. Current methodology requires visual comparison of preoperative diagnostic imaging with real-time procedural imaging, often requiring subjective comparison of cross-sectional imaging to IOUS. Then, manual free-hand IOUS is employed in conjunction with free-hand positioning of the tissue ablator under ultrasound guidance. Target motion upon insertion of the ablation probe makes it difficult to localize appropriate placement of the therapy device with simultaneous target imaging. The major limitation of ablative approaches is the lack of accuracy in probe localization within the center of the tumor. This is particularly important, as histological margins cannot be assessed after ablations as opposed to hepatic resection approaches [Koniaris-2000] [Scott-2001]. In addition, manual guidance often requires multiple passes and repositioning of the ablator tip, further increasing the risk of bleeding and tumor dissemination. In situations when the desired target zone is larger than the single ablation size (e.g. 5-cm tumor and 4-cm ablation device), multiple overlapping spheres are required in order to achieve complete tumor destruction. In such cases, the capacity to accurately plan multiple manual ablations is significantly impaired by the complex 3D geometrically complex planning required as well as image distortion artifacts from the first ablation, further reducing the targeting confidence and potential efficacy of the therapy. IOUS often provides excellent visualization of tumors and guidance for probe placement, but its 2D-nature and dependence on the sonographer's skills limit its effectiveness [Wood-2000].

Improved real-time guidance for planning, delivery and monitoring of the ablative therapy would provide the missing tool needed to enable accurate and effective application of this promising therapy. Recent studies are beginning to identify reasons for diminished efficacy of ablative approaches, including size, location, operator experience, and technical approach [Mulier-2005] [van Duijnhoven-2006]. These studies suggest that device targeting and ablation monitoring are likely the key reasons for local failure. Also, due to gas bubbles, bleeding, or edema, IOUS images provide limited visualization of tumor margins or even the applicator electrode position during RFA [Hinshaw-2007].

The impact of radiological complete response on tumor targeting is an important emerging problem in liver directed therapy. Specifically, this problem relates to the inability to identify the target tumor at the time of therapy. Effective combination systemic chemotherapeutic regimens are being used with increasing frequency prior to liver-directed therapy to treat potential micro-metastatic disease as a neo-adjuvant approach, particularly for colorectal metastases [Gruenberger-2008]. This allows the opportunity to use the liver tumor as a gauge to determine chemo-responsiveness as an aid to planning subsequent post-procedural chemotherapy. However, in such an approach, the target lesion often cannot be identified during the subsequent resection or ablation. We know that even when the index liver lesion is no longer visible, microscopic tumors are still present in more than 80% of cases [Benoist-2006]. Any potentially curative approach, therefore, still requires complete resection or local destruction of all original sites of disease. In such cases, the interventionalist can face the situation of contemplating a “blind” ablation in region of the liver in which no imagable tumor can be detected. Therefore, without an ability to identify original sites of disease, preoperative systemic therapies may actually hinder the ability to achieve curative local targeting, paradoxically potentially worsening long-term survival. As proposed in this project, integrating a strategy for registration of the pre-chemotherapy cross-sectional imaging (CT) with the procedure-based imaging (IOUS) would provide invaluable information for ablation guidance.

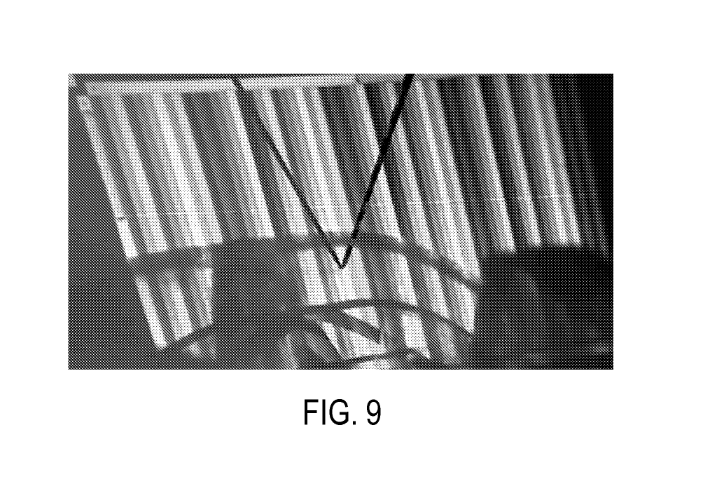

Our system embodiments described both in FIG. 1 and FIG. 2 can be utilized in the above mentioned application. With structured light attached to the ultrasound probe, patient surface can be captured and digitized in real-time. Then, the doctor will select an area of interest to scan where he/she can observe a lesion either directly from the ultrasound images or indirectly from the fused pre-operative data. The fusion is performed by integrating both surface data from structured light and few ultrasound images and can be updated in real-time without manual input from the user. Once the lesion is identified in the US probe space, the doctor can introduce the ablation probe, where the SLS system can easily segment/track and localize the tool before inserting to the patient (FIG. 9). The projector can be used to overlay real-time guidance information to help orient the tool and provide a feedback about the needed insertion depth.

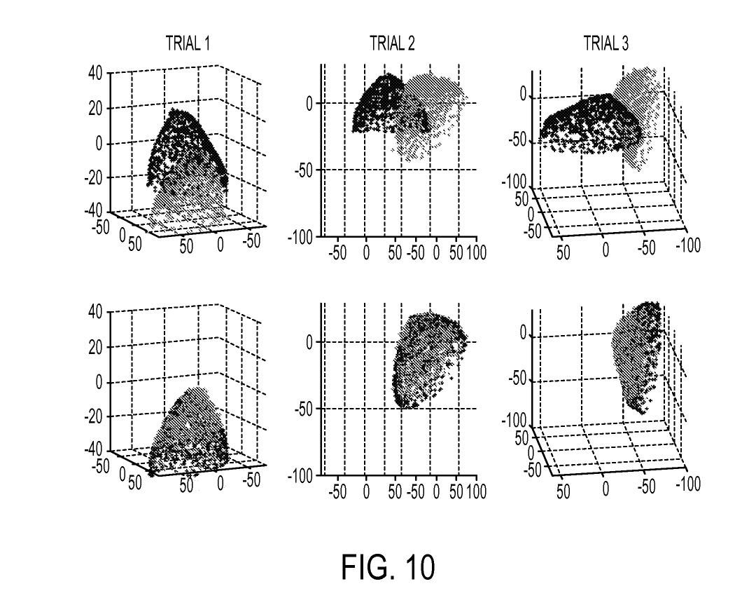

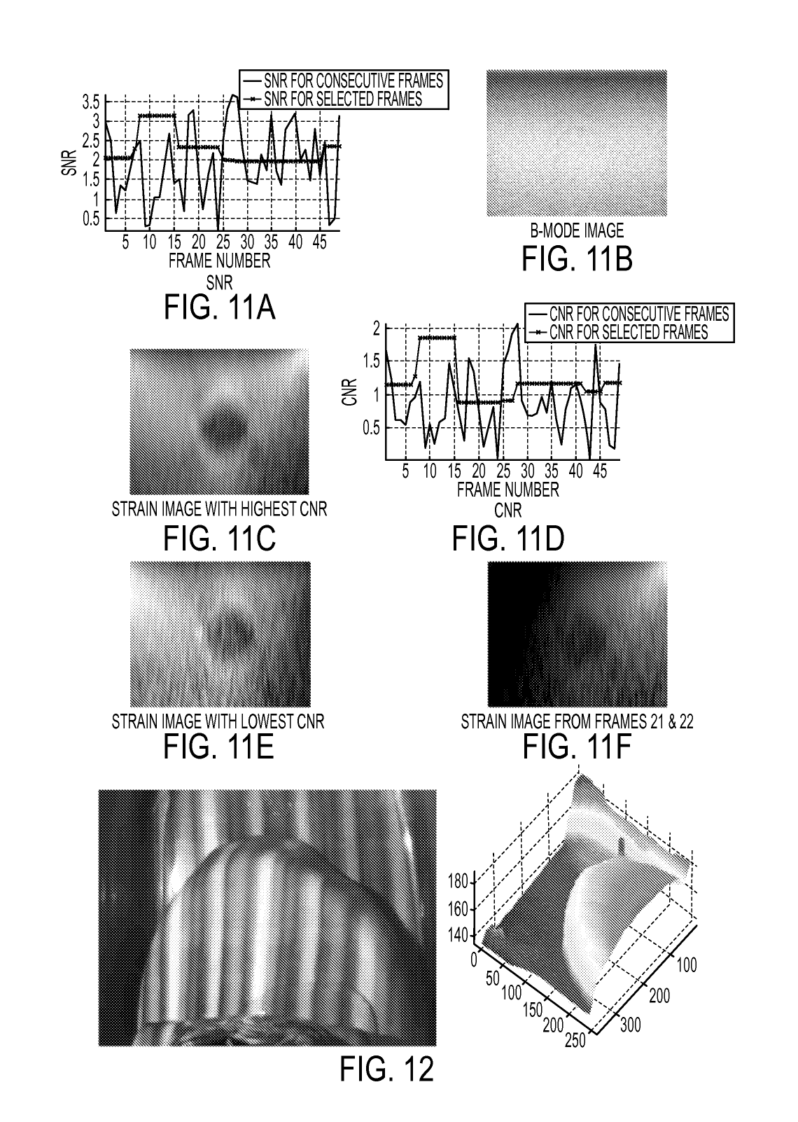

Abovementioned is the embodiment described in FIG. 1. However, our invention includes many alternates for example: 1) Time-of-flight camera can replace the SLS configuration to provide the surface data [Billings-2011] (FIG. 10). In this embodiment, the ToF camera is not attached to the ultrasound probe, and an external tracker is used to track both components. Projector can still be attached to the ultrasound probe. 2) Another embodiment consists of SLS or ToF camera to provide surface information and a projector attached to the ultrasound probe. The camera configuration, i.e. SLS should be able to extract surface data, track intervention tool, and probe surface, hence can locate the needle to the US image coordinate. This embodiment requires offline calibration to estimate the transformation between the probe surface shape and the actual location of the ultrasound image. A projector still can be used to overlay needle location and visualize guidance information. 3) Furthermore, embodiment can only consist of projectors and local sensors. FIG. 7 describes a system composed of pulsed laser projector to track an interventional tool in air and in tissue using photoacoustic (PA) phenomenon [Boctor-2010]. Interventional tools can convert pulsed light energy into an acoustic wave that can be picked up by multiple acoustic sensors placed on the probe surface, which we then can apply known triangulation algorithms to locate the needle. It is important to note that one can apply laser light directly to the needle, i.e. attach fiber optic configuration to a needle end; the needle can also conduct the generated acoustic wave (i.e. acting like a wave-guide) and fraction of this acoustic wave can propagate from the needle shaft and tip and the PA signals, i.e. acoustic signals generated, can be picked up by both sensors attached to the surface as well as the ultrasound array elements. In addition to the laser light projecting directly to the needle, we can extend few fibers to deposit light energy underneath the probe, hence can track the needle inside the tissue (FIG. 7).

One possible embodiment is to integrate both an ultrasound probe with an endoscopic camera held on one endoscopic channel and having the projector component connected in a separate channel. This projector can enable structured light, and the endoscopic camera performs surface estimation to help performing hybrid surface/ultrasound registration with a pre-operative modality. Possibly, the projector can be a pulsed laser projector that can enable PA effects and the ultrasound probe attached to the camera can generate PA images for region of interest.

### REFERENCES

[Benoist-2006] Benoist S, Brouquet A, Penna C, Julié C, El Hajjam M, Chagnon S, Mitry E, Rougier P, Nordlinger B, “Complete response of colorectal liver metastases after chemotherapy: does it mean cure?” J Clin Oncol. 2006 Aug. 20; 24(24):3939-45.
[Berber-2008] Berber E, Tsinberg M, Tellioglu G, Simpfendorfer C H, Siperstein A E. Resection versus laparoscopic radiofrequency thermal ablation of solitary colorectal liver metastasis. J Gastrointest Surg. 2008 November; 12(11):1967-72.
[Billings-2011] Billings S, Kapoor A, Wood B J, Boctor E M, “A hybrid surface/image based approach to facilitate ultrasound/CT registration,” accepted SPIE Medical Imaging 2011.
[Boctor-2010] E. Boctor, S. Verma et al. “Prostate brachytherapy seed localization using combined photoacoustic and ultrasound imaging,” SPIE Medical Imaging 2010.
[Chen-2006] Chen M S, Li J Q, Zheng Y, Guo R P, Liang H H, Zhang Y Q, Lin X J, Lau W Y. A prospective randomized trial comparing percutaneous local ablative therapy and partial hepatectomy for small hepatocellular carcinoma. Ann Surg. 2006 March; 243(3):321-8.
[Goldberg-2000] Goldberg S N, Gazelle G S, Mueller P R. Thermal ablation therapy for focal malignancy: a unified approach to underlying principles, techniques, and diagnostic imaging guidance. AJR Am J. Roentgenol. 2000 February; 174(2):323-31.
[Gruenberger-2008] Gruenberger B, Scheithauer W, Punzengruber R, Zielinski C, Tamandl D, Gruenberger T. Importance of response to neoadjuvant chemotherapy in potentially curable colorectal cancer liver metastases. BMC Cancer. 2008 Apr. 25; 8:120.
[Hinshaw-2007] Hinshaw J L, et. al., Multiple-Electrode Radiofrequency Ablation of Symptomatic Hepatic Cavernous Hemangioma, Am. J. Roentgenol., Vol. 189, Issue 3, W-149, Sep. 1, 2007.
[Koichi-2008] Koichi O, Nobuyuki M, Masaru O et al., “Insufficient radiofrequency ablation therapy may induce further malignant transformation of hepatocellular carcinoma,” Journal of Hepatology International, Volume 2, Number 1, March 2008, pp 116-123.
[Koniaris-2000] Koniaris L G, Chan D Y, Magee C, Solomon S B, Anderson J H, Smith D O, DeWeese T, Kavoussi L R, Choti M A, “Focal hepatic ablation using interstitial photon radiation energy,” J Am Coll Surg. 2000 August; 191(2):164-74.
[Mulier-2005] Mulier S, Ni Y, Jamart J, Ruers T, Marchal G, Michel L. Local recurrence after hepatic radiofrequency coagulation: multivariate meta-analysis and review of contributing factors. Ann Surg. 2005 August; 242(2):158-71.
[Poon-2004] Poon R T, Ng K K, Lam C M, Ai V, Yuen J, Fan S T, Wong J. Learning curve for radiofrequency ablation of liver tumors: prospective analysis of initial 100 patients in a tertiary institution. Ann Surg. 2004 April; 239(4):441-9.
[Scott-2001] Scott D J, Young W N, Watumull L M, Lindberg G, Fleming J B, Huth J F, Rege R V, Jeyarajah D R, Jones D B, “Accuracy and effectiveness of laparoscopic vs open hepatic radiofrequency ablation,” Surg Endosc. 2001 February; 15(2):135-40.
[van Duijnhoven-2006] van Duijnhoven F H, Jansen M C, Junggeburt J M, van Hillegersberg R, Rijken A M, van Coevorden F, van der Sijp J R, van Gulik T M, Slooter G D, Klaase J M, Putter H, Tollenaar R A, “Factors influencing the local failure rate of radiofrequency ablation of colorectal liver metastases,” Ann Surg Oncol. 2006 May; 13(5):651-8. Epub 2006 March 17.
[Wood-2000] Wood T F, Rose D M, Chung M, Allegra D P, Foshag L J, Bilchik A J, “Radiofrequency ablation of 231 unresectable hepatic tumors: indications, limitations, and complications,” Ann Surg Oncol. 2000 September; 7(8):593-600.

### Example 2

### Monitoring Neo-Adjuvant Chemotherapy Using Advanced Ultrasound Imaging

Out of more than two hundred thousand women diagnosed with breast cancer every year, about 10% will present with locally advanced disease [Valero-1996]. Primary chemotherapy (a.k.a. Neo-adjuvant chemotherapy, NAC) is quickly replacing adjuvant (post-operative) chemotherapy as the standard in the management of these patients. In addition, NAC is often administered to women with operable stage II or III breast cancer [Kaufmann-2006]. The benefit of NAC is two fold. First, NAC has the ability to increase the rate of breast conserving therapy. Studies have shown that more than fifty percent of women, who would otherwise be candidates for mastectomy only, become eligible for breast conserving therapy because of NAC induced tumor shrinkage [Hortabagyi-1988, Bonadonna-1998]. Second, NAC allows in vivo chemo-sensitivity assessment. The ability to detect early drug resistance will prompt change from the ineffective to an effective regimen. Consequently, physicians may decrease toxicity and perhaps improve outcome. The metric most commonly used to determine in-vivo efficacy is the change in the tumor sized during NAC.

Unfortunately, the clinical tools used to measure tumor size during NAC, such as physical exam, mammography, and B-mode ultrasound, have been shown to be less than ideal. Researchers have shown that post-NAC tumor size estimates by physical exam, ultrasound and mammography, when compared to pathologic measurements, have correlation coefficients of 0.42, 0.42, and 0.41 respectively [Chagpar-2006]. MRI and PET appear to be more predictive of response to NAC however these modalities are expensive, inconvenient and, with respect to PET, impractical for serial use due to excessive radiation exposure [Smith-2000, Rosen-2003, Partridge-2002]. What is needed is an inexpensive, convenient and safe technique capable of accurately measuring tumor response repeatedly during NAC.

Ultrasound is a safe modality which easily lends itself to serial use. However, the most common system currently in medical use, B-Mode ultrasound, does not appear to be sensitive enough to determine subtle changes in tumor size. Accordingly, USEI has emerged as a potentially useful augmentation to conventional ultrasound imaging. USEI has been made possible by two discoveries: (1) different tissues may have significant differences in their mechanical properties and (2) the information encoded in the coherent scattering (a.k.a. speckle) may be sufficient to calculate these differences following a mechanical stimulus [Ophir-1991]. An array of parameters, such as velocity of vibration, displacement, strain, velocity of wave propagation and elastic modulus, have been successfully estimated [Konofagou-2004, Greenleaf-2003], which then made it possible to delineate stiffer tissue masses, such as tumors [Hall-2002, Lyshchik-2005, Purohit-2003], ablated lesions [Varghese-2004, Boctor-2005]. Breast cancer detection is the first [Garra-1997] and most promising [Hall-2003] application of USEI.

An embodiment for this application is to use an ultrasound probe and an SLS configuration attached to the external passive arm. We can track both the SLS and the ultrasound probe using external tracking device, or simply use the SLS configuration to track the probe with respect to SLS's own reference frame. On day one, we place the probe one the region of interest and the SLS configuration captures the breast surface information, the ultrasound probe surface and provides a substantial input for the following task: 1) The US probe can be tracked and hence 3D US volume can be reconstructed from 2D images (the US probe is a 2D probe); or the resulting small volumes from a 3D probe can be stitched together and form a panoramic volume, 2). The US probe can be tracked during elastography scan. This tracking information can be integrated in the EI algorithm to enhance the quality [Foroughi-2010] (FIG. 11), and 3) Registration between ultrasound probe's location on the first treatment session and subsequent sessions can be easily recovered using the SLS surface information (as shown in FIG. 12) for both the US probe and the breast.

### REFERENCES

[Boctor-2005] Boctor E M, DeOliviera. M, Awad M., Taylor R H, Fichtinger G, Choti M A, Robot-assisted 3D strain imaging for monitoring thermal ablation of liver, Annual congress of the Society of American Gastrointestinal Endoscopic Surgeons, pp 240-241, 2005.
[Bonadonna-1998] Bonadonna G, Valagussa P, Brambilla C, Ferrari L, Moliterni A, Terenziani M, Zambetti M, “Primary chemotherapy in operable breast cancer: eight-year experience at the Milan Cancer Institute,” SOJ Clin Oncol 1998 January; 16(1):93-100.
[Chagpar-2006] Chagpar A, et al., “Accuracy of Physical Examination, Ultrasonography and Mammography in Predicting Residual Pathologic Tumor size in patients treated with neoadjuvant chemotherapy” Annals of surgery Vol. 243, Number 2, February 2006.
[Greenleaf-2003] Greenleaf J F, Fatemi M, Insana M. Selected methods for imaging elastic properties of biological tissues. Annu Rev Biomed Eng. 2003; 5:57-78.
[Hall-2002] Hall T J, Yanning Zhu, Spalding C S “In vivo real-time freehand palpation imaging Ultrasound Med Biol. 2003 March; 29(3):427-35.
[Konofagou-2004] Konofagou E E. Quovadis elasticity imaging? Ultrasonics. 2004 April; 42(1-9):331-6.
[Lyshchik-2005] Lyshchik A, Higashi T, Asato R, Tanaka S, Ito J, Mai J J, Pellot-Barakat C, Insana M F, Brill A B, Saga T, Hiraoka M, Togashi K. Thyroid gland tumor diagnosis at US elastography. Radiology. 2005 October; 237(1):202-11.
[Ophir-1991] Ophir J, Céspedes E I, Ponnekanti H, Yazdi Y, Li X: Elastography: a quantitative method for imaging the elasticity of biological tissues. Ultrasonic Imag., 13:111-134, 1991.
[Partridge-2002] Partridge S C, Gibbs J E, Lu Y, Esserman L J, Sudilovsky D, Hylton N M, “Accuracy of MR imaging for revealing residual breast cancer in patients who have undergone neoadjuvant chemotherapy,” AJR Am J. Roentgenol. 2002 November; 179(5):1193-9.
[Purohit-2003] Purohit R S, Shinohara K, Meng M V, Carroll P R. Imaging clinically localized prostate cancer. Urol Clin North A m. 2003 May; 30(2):279-93.
[Rosen-2003] Rosen E L, Blackwell K L, Baker J A, Soo M S, Bentley R C, Yu D, Samulski T V, Dewhirst M W, “Accuracy of MRI in the detection of residual breast cancer after neoadjuvant chemotherapy,” AJR Am J. Roentgenol. 2003. November; 181(5): 1275-82.
[Smith-2000] Smith I C, Welch A E, Hutcheon A W, Miller I D, Payne S, Chilcott F, Waikar S, Whitaker T, Ah-See A K, Eremin O, Heys S D, Gilbert F J, Sharp P F, “Positron emission tomography using [(18)F]-fluorodeoxy-D-glucose to predict the pathologic response of breast cancer to primary chemotherapy,” J Clin Oncol. 2000 April; 18(8):1676-88.
[Valero-1996] Valero V, Buzdar A U, Hortobagyi G N, “Locally Advanced Breast Cancer,” Oncologist. 1996; 1(1 & 2):8-17.
[Varghese-2004] Varghese T, Shi H. Elastographic imaging of thermal lesions in liver in-vivo using diaphragmatic stimuli. Ultrason Imaging. 2004 January; 26(1):18-28.
[Foroughi-2010] P. Foroughi, H. Rivaz, I. N. Fleming, G. D. Hager, and E. Boctor, “Tracked Ultrasound Elastography (TrUE),” in Medical Image Computing and Computer Integrated surgery, 2010.

### Example 3

### Ultrasound Imaging Guidance for Laparoscopic Partial Nephrectomy

Kidney cancer is the most lethal of all genitourinary tumors, resulting in greater than 13,000 deaths in 2008 out of 55,000 new cases diagnosed [61]. Further, the rate at which kidney cancer is diagnosed is increasing [1,2,62]. “Small” localized tumors currently represent approximately 66% of new diagnoses of renal cell carcinoma [63].

Surgery remains the current gold standard for treatment of localized kidney tumors, although alternative therapeutic approaches including active surveillance and emerging ablative technologies [5] exist. Five year cancer-specific survival for small renal tumors treated surgically is greater than 95% [3,4]. Surgical treatments include simple nephrectomy (removal of the kidney), radical nephrectomy (removal of the kidney, adrenal gland, and some surrounding tissue) and partial nephrectomy (removal of the tumor and a small margin of surrounding tissue, but leaving the rest of the kidney intact). More recently, a laparoscopic option for partial nephrectomy (LPN) has been developed with apparently equivalent cancer control results compared to the open approach [9,10]. The benefits of the laparoscopic approach are improved cosmesis, decreased pain, and improved convalescence relative to the open approach.

Although a total nephrectomy will remove the tumor, it can have serious consequences for patients whose other kidney is damaged or missing or who are otherwise at risk of developing severely compromised kidney function. This is significant given the prevalence of risk factors for chronic renal failure such as diabetes and hypertension in the general population [7,8]. Partial nephrectomy has been shown to be oncologically equivalent to total nephrectomy removal for treatment of renal tumors less than 4 cm in size (e.g., [3,6]). Further, data suggest that patients undergoing partial nephrectomy for treatment of their small renal tumor enjoy a survival benefit compared to those undergoing radical nephrectomy [12-14]. A recent study utilizing the Surveillance, Epidemiology and End Results cancer registry identified 2,991 patients older than 66 years who were treated with either radical or partial nephrectomy for renal tumors<4 cm [12]. Radical nephrectomy was associated with an increased risk of overall mortality (HR 1.38, p<0.01) and a 1.4 times greater number of cardiovascular events after surgery compared to partial nephrectomy.

Despite the advantages in outcomes, partial nephrectomies are performed in only 7.5% of cases [11]. One key reason for this disparity is the technical difficulty of the procedure. The surgeon must work very quickly to complete the resection, perform the necessary anastamoses, and restore circulation before the kidney is damaged. Further, the surgeon must know where to cut to ensure cancer-free resection margins while still preserving as much good kidney tissue as possible. In performing the resection, the surgeon must rely on memory and visual judgment to relate preoperative CT and other information to the physical reality of the patient's kidney. These difficulties are greatly magnified when the procedure is performed laparoscopically, due to the reduced dexterity associated with the instruments and reduced visualization from the laparoscope.

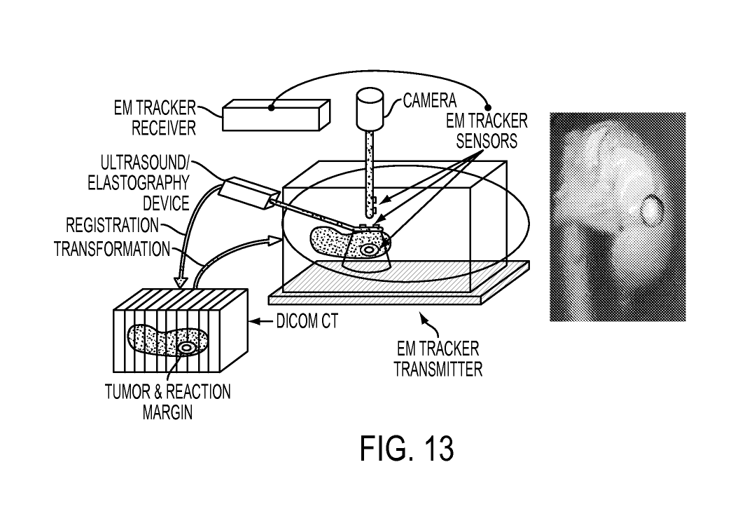

We devised two embodiments to overcome this technically challenging intervention. FIG. 13 shows the first system where an SLS component is held on a laparoscopic arm, a laparoscopic ultrasound probe and an external tracking device to track both the US probe and the SLS [Stolka-2010]. However, we don't need to rely on an external tracking device since we have access to an SLS configuration. SLS can scan kidney surface and probe surface and track both kidney and the US probe. Furthermore, our invention is concerned with Hybrid surface/ultrasound registration. In this embodiment the SLS will scan the kidney surface and together with few ultrasound images a reliable registration with pre-operative data can be performed and augmented visualization, similar to the one shown in FIG. 13, can be visualized using the attached projector.

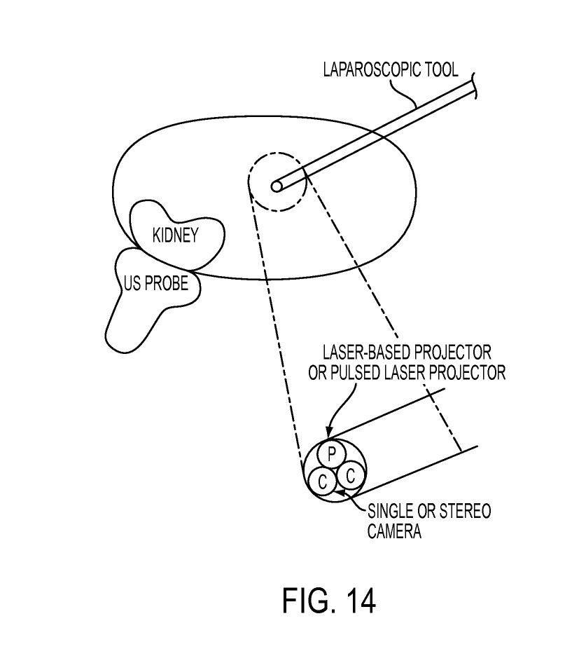

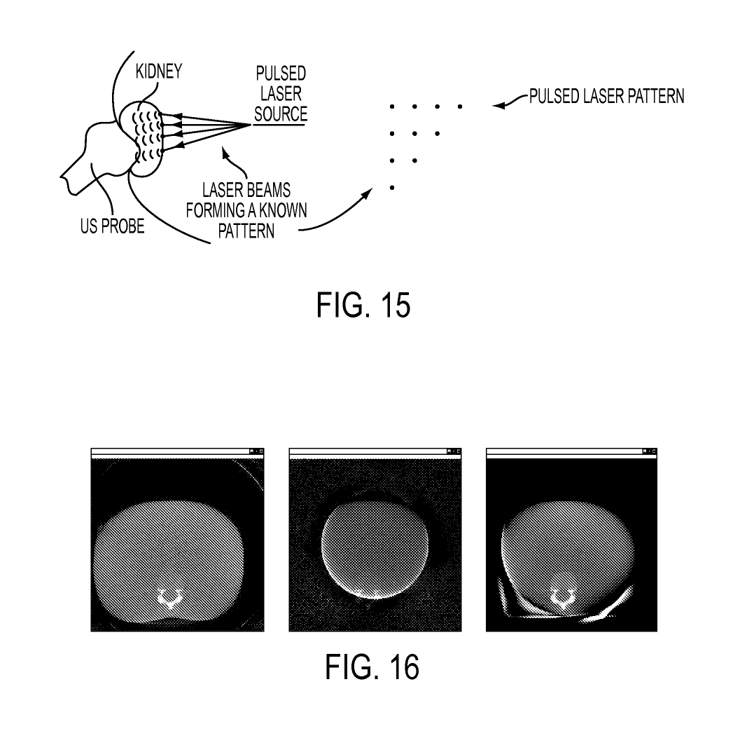

The second embodiment is shown in FIG. 14 where an ultrasound probe is located outside the patient and facing directly towards the superficial side of the kidney. Internally a laparoscopic tool holds an SLS configuration. The SLS system provides kidney surface information in real-time and the 3DUS also images the same surface (tissue-air interface). By applying surface-to-surface registration ultrasound volume can be easily registered to the SLS reference frame. In a different embodiment, registration can be also performed using photoacoustic effect (FIG. 15). Typically, the project in the SLS configuration can be a pulsed laser, projector with a fixed pattern. Photoacoustic signals will be generated at specified points, which forms a known calibrated pattern. The ultrasound imager can detect these points PA signals. Then a straightforward point-to-point registration can be performed to establish real-time registration between the camera/projector-space and the ultrasound space.

C-Arm-Guided Interventional Application

Projection data truncation problem is a common issue with reconstructed CT and C-arm images. This problem appears clearly near the image boundaries. Truncation is a result of the incomplete data set obtained from the CT/C-arm modality. An algorithm to overcome this truncation error has been developed [Xu-2010]. In addition to the projection data, this algorithm requires the patient contour in 3D space with respect to the X-Ray detector. This contour is used to generate the trust region required to guide the reconstruction method. A simulation study on a digital phantom was done [Xu-2010] to reveal the enhancement achieved by the new method. However, a practical way to get the trust region has to be developed. FIG. 3 and FIG. 4 present novel practical embodiments to track and to obtain the patient contour information and consequentially the trust region at each view angle of the scan. The trust region is used to guide the reconstruction method [Ismail-2011].

It is known that X-ray is not ideal modality for soft-tissue imaging. Recent C-arm interventional systems are equipped with flat-panel detectors and can perform cone-beam reconstruction. The reconstruction volume can be used to register intraoperative X-ray data to pre-operative MRI. Typically, couple of hundreds X-ray shots need to be taken in order to perform the reconstruction task. Our novel embodiments are capable of performing surface-to-surface registration by utilizing real-time and intraoperative surfaces from SLS or ToF or similar surface scanner sensors. Hence, reducing X-ray dosage is achieved. Nevertheless, if there is need to fine tune the registration task, in this case few X-rays images can be integrated in the overall framework.

It is obvious that similar to US navigation examples and methods described before, the SLS component configured and calibrated to a C-arm can also track interventional tools and the projector attached can provide real-time visualization.

Furthermore, ultrasound probe can be easily introduced to the C-arm scene without adding or changing the current setup. The SLS configuration is capable of tracking the US probe. It is important to note that in many pediatric interventional applications, there is need to integrate ultrasound imager to the C-arm suite. In these scenarios, the SLS configuration can be either attached to the C-arm, to the ultrasound probe, or separately attached to an arm. This ultrasound/C-arm system can consist of more than one SLS configuration, or combination of these sensors. For example, the camera or multiple cameras can be fixed to the C-arm where the projector can be attached to the US probe.

Finally, our novel embodiment can provide quality control to the C-arm calibration. C-arm is a moving equipment and can't be considered a rigid-body, i.e. there is a small rocking/vibrating motion that need to be measured/calibrated at the manufacture site and these numbers are used to compensate during reconstruction. If a faulty condition happened that alter this calibration, the company needs to be informed to re-calibrate the system. These faulty conditions are hard to detect and repeated QC calibration is also unfeasible and expensive. Our accurate surface tracker should be able to determine the motion of the C-arm and continuously, in the background, compare to the manufacture calibration. Once a faulty condition happens, our system should be able to discover and possible correct it.

### REFERENCES

[Jemal-2007] Jemal A, Siegel R, Ward E, Murray T, Xu J, Thun M J. Cancer statistics, 2007. CA Cancer J Clin 2007 January-February; 57(1):43-66.
2. [Volpe-2004] Volpe A, Panzarella T, Rendon R A, Haider M A, Kondylis F I, Jewett M A. The natural history of incidentally detected small renal masses. Cancer 2004 Feb. 15; 100(4):738-45
3. [Fergany-2000] Fergany A F, Hafez K S, Novick A C. Long-term results of nephron sparing surgery for localized renal cell carcinoma: 10-year followup. J Urol 2000 February; 163(2):442-5.
4. [Hafez-1999] Hafez K S, Fergany A F, Novick A C. Nephron sparing surgery for localized renal cell carcinoma: impact of tumor size on patient survival, tumor recurrence and TNM staging. J Urol1999 December; 162(6):1930-3.
5. [Kunkle-2008] Kunkle D A, Egleston B L, Uzzo R G. Excise, ablate or observe: the small renal mass dilemma—a meta-analysis and review. J Urol2008 April; 179(4):1227-33; discussion-33-4.
6. [Leibovich-2004] Leibovich B C, Blute M L, Cheville J C, Lohse C M, Weaver A L, Zincke H. Nephron sparing surgery for appropriately selected renal cell carcinoma between 4 and 7 cm results in outcome similar to radical nephrectomy. J Urol2004 March; 171(3):1066-70.
7. [Coresh-2007] Coresh J, Selvin E, Stevens L A, Manzi J, Kusek J W, Eggers P, et al. Prevalence of chronic kidney disease in the United States. JAMA2007 Nov. 7; 298(17):2038-47.
8. [Bijol-2006] Bijol V, Mendez G P, Hurwitz S, Rennke H G, Nose V. Evaluation of the normeoplastic pathology in tumor nephrectomy specimens: predicting the risk of progressive renal failure. Am J Surg Pathol 2006 May; 30(5):575-84.
9. [Allaf-2004] Allaf M E, Bhayani S B, Rogers C, Varkarakis I, Link R E, Inagaki T, et al. Laparoscopic partial nephrectomy: evaluation of long-term oncological outcome. J Urol2004 September; 172(3):871-3.
10. [Moinzadeh-2006] Moinzadeh A, Gill I S, Finelli A, Kaouk J, Desai M. Laparoscopic partial nephrectomy: 3-year followup. J Urol2006 February; 175(2):459-62.
11. [Hollenbeck-2006] Hollenbeck B K, Taub D A, Miller D C, Dunn R L, Wei J T. National utilization trends of partial nephrectomy for renal cell carcinoma: a case of underutilization? Urology2006 February; 67(2):254-9.
12. [Huang-2009] Huang W C, Elkin E B, Levey A S, Jang T L, Russo P. Partial nephrectomy versus radical nephrectomy in patients with small renal tumors—is there a difference in mortality and cardiovascular outcomes? J Urol2009 January; 181(1):55-61; discussion—2.
13. [Thompson-2008] Thompson R H, Boorjian S A, Lohse C M, Leibovich B C, Kwon E D, Cheville J C, et al. Radical nephrectomy for pT1a renal masses may be associated with decreased overall survival compared with partial nephrectomy. J Urol 2008 February; 179(2):468-71; discussion 72-3.
14. [Zini-2009] Zini L, Perrotte P, Capitanio U, Jeldres C, Shariat S F, Antebi E, et al. Radical versus partial nephrectomy: effect on overall and noncancer mortality. Cancer2009 Apr. 1; 115(7):1465-71.
15. Stolka P J, Keil M, Sakas G, McVeigh E R, Taylor R H, Boctor E M, “A 3D-elastography-guided system for laparoscopic partial nephrectomies”. SPIE Medical Imaging 2010 (San Diego, Calif./USA)
61. [Jemal-2008] Jemal A, Siegel R, Ward E, et al. Cancer statistics, 2008. CA Cancer J Clin 2008; 58:71-96. SFX
62. [Hock-2002] Hock L, Lynch J, Balaji K. Increasing incidence of all stages of kidney cancer in the last 2 decades in the United States: an analysis of surveillance, epidemiology and end results program data. J Urol 2002; 167:57-60. Ovid Full Text Bibliographic Links
63. [Volpe-2005] Volpe A, Jewett M. The natural history of small renal masses. Nat Clin Pract Urol 2005; 2:384-390. SFX
[Ismail-2011] Ismail M M, Taguchi K, Xu J, Tsui B M, Boctor E, “3D-guided CT reconstruction using time-of-flight camera,” Accepted in SPIE Medical Imaging 2011
[Xu-2010] Xu, J.; Taguchi, K.; Tsui, B. M. W.; “Statistical Projection Completion in X-ray CT Using Consistency Conditions,” Medical Imaging, IEEE Transactions on, vol. 29, no. 8, pp. 1528-1540, August 2010

1. An augmentation device for an imaging system, comprising:
a bracket structured to be attachable to an imaging component; and
a projector attached to said bracket,
wherein said projector is arranged and configured to project an image onto a surface in conjunction with imaging by said imaging system.

2. (canceled)

3. An augmentation device according to claim 1, further comprising a camera attached to said bracket.

4. (canceled)

5. An augmentation device according to claim 3, further comprising a second camera attached to said bracket.

6. An augmentation device according to claim 5, wherein the first-mentioned camera is arranged to observe a region of imaging during operation of said imaging system and said second camera is at least one of arranged to observe said region of imaging to provide stereo viewing or to observe a user during imaging to provide information regarding a viewing position of said user.

7. An augmentation device according to claim 1, further comprising a local sensor system attached to said bracket, wherein said local sensor system provides at least one of position and orientation information of said imaging component to permit tracking of said imaging component while in use.

8. An augmentation device according to claim 3, further comprising a local sensor system attached to said bracket, wherein said local sensor system provides at least one of position and orientation information of said imaging component to permit tracking of said imaging component while in use.

9. An augmentation device according to claim 7, wherein said local sensor system comprises at least one of an optical, inertial or capacitive sensor.

10. An augmentation device according to claim 7, wherein said local sensor system comprises a three-axis gyro system that provides rotation information about three orthogonal axes of rotation.

11. (canceled)

12. An augmentation device according to claim 10, wherein said local sensor system comprises a system of linear accelerometers that provide acceleration information along at least two orthogonal axes.

13. (canceled)

14. An augmentation device according to claim 8, wherein said local sensor system comprises an optical sensor system arranged to detect motion of said imaging component with respect to a surface.

15. An augmentation device according to claim 12, wherein said imaging system is a component of an image-guided surgery system.

16. An augmentation device according to claim 15, wherein said imaging system is an ultrasound imaging system and said imaging component is an ultrasound probe handle, said bracket being structured to be attachable to said ultrasound probe handle.

17. (canceled)

18. An augmentation device according to claim 3, further comprising a second camera attached to said bracket, wherein the first-mentioned and second cameras are arranged and configured to provide stereo viewing of a region of interest during imaging with said imaging system, wherein said projector is configured and arranged to project a pattern on a surface in view of the first-mentioned and said second cameras to facilitate stereo object recognition and tracking of objects in view of said cameras.

19. (canceled)

20. (canceled)

21. An augmentation device according to claim 7, further comprising a communication system in communication with at least one of said local sensor system, said camera or said projector.

22. (canceled)

23. A system for image-guided surgery, comprising:
an imaging system; and
a projector configured to project an image onto a region of interest during imaging by said imaging system.

24. (canceled)

25. (canceled)

26. (canceled)

27. A system for image-guided surgery according to claim 23, further comprising a camera arranged to capture an image of a second region of interest during imaging by said imaging system.

28. (canceled)

29. (canceled)

30. A system for image-guided surgery according to claim 27, further comprising a second camera arranged to capture an image of a third region of interest during imaging by said imaging system.

31. A system for image-guided surgery according to claim 30, further comprising a sensor system comprising a component attached to at least one of said imaging system, said projector, the first-mention camera, or said second camera, wherein said sensor system provides at least one of position and orientation information of said imaging system, said projector, the first-mention camera, or said second camera to permit tracking while in use.

32. A system for image-guided surgery according to claim 31, wherein said sensor system is a local sensor system providing tracking free from external reference frames.

33. A system for image-guided surgery according to claim 32, wherein said local sensor system comprises at least one of an optical, inertial or capacitive sensor.

34. A system for image-guided surgery according to claim 32, wherein said local sensor system comprises a three-axis gyro system that provides rotation information about three orthogonal axes of rotation.

35. (canceled)

36. A system for image-guided surgery according to claim 32, wherein said local sensor system comprises a system of linear accelerometers that provide acceleration information along at least two orthogonal axes.

37. (canceled)

38. A system for image-guided surgery according to claim 32, wherein said local sensor system comprises an optical sensor system arranged to detect motion of said imaging component with respect to a surface.

39. A system for image-guided surgery according to claim 32, further comprising a communication system in communication with at least one of said local sensor system, said camera or said projector.

40. A system for image-guided surgery according to claim 39, wherein said communication system is a wireless communication system.

41. A capsule imaging device, comprising:
an imaging system; and
a local sensor system,
wherein said local sensor system provides information to reconstruct positions of said capsule endoscope free from external monitoring equipment.

42. A capsule imaging device according to claim 41, wherein said imaging system is an optical imaging system.

43. A capsule imaging device according to claim 41, wherein said imaging system is an ultrasound imaging system.

44. A capsule imaging device according to claim 43, wherein said ultrasound imaging system comprises a pulsed laser and an ultrasound receiver configured to detect ultrasound signals in response to pulses from said pulsed laser interacting with material in regions of interest.

45. A system for image-guided surgery according to claim 31, further compromising a projection screen that is adapted to be at least one of a handheld or attached to a component of said system.

46. (canceled)
